(19) United States
(12) Patent Application Publication
(10) Pub. No.: US 2018/0230553 A1
(43) Pub. Date: Aug. 16, 2018

(54) METHODS FOR PREDICTING PALM OIL YIELD OF A TEST OIL PALM PLANT

(71) Applicant: SIME DARBY PLANTATION BERHAD, Selangor (MY)

(72) Inventors: Nurliyana Ruzlan, Serdang (MY); Ainul Masni Othman, Serdang (MY); Wilonita Win, Serdang (MY); Shwu Fun Kua, Serdang (MY); Jaime Yoke Sum Low, Serdang (MY); Anusha Nair Chandran, Serdang (MY); Li Sim Ho, Serdang (MY); Norazura Anuar, Serdang (MY); Bee Aik Tan, Serdang (MY); Noor Azizah Musa, Serdang (MY); Ai Ling Ong, Serdang (MY); Hirzun Mohd Yusof, Serdang (MY); David Ross Appleton, Serdang (MY); Harikrishna Kulaveerasingam, Carey Island (MY)

(21) Appl. No.: 15/750,446

(22) PCT Filed: May 26, 2016
(86) PCT No.: PCT/MY2016/000032

§ 371 Date: Feb. 5, 2018

(30) Foreign Application Priority Data
Aug. 6, 2015 (MY) PI 2015702583

(51) Int. Cl. C12Q 1/6895

(52) U.S. Cl. CPC C12Q 1/6895; C12Q 2600/156; C12Q 2600/13

(57) ABSTRACT

Methods for predicting palm oil yield of a test oil palm plant are disclosed. The methods comprise determining, from a sample of a test oil palm plant of a population, at least a first SNP genotype, corresponding to a first SNP marker, located in a first candidate gene region for a high-oil-production trait and associated, after stratification and kinship correction, with the high-oil-production trait with a p-value <0.001 in the population or having a linkage disequilibrium r² value of at least 0.2 with respect to a first other SNP marker that is linked thereto and associated, after stratification and kinship correction, with the high-oil-production trait a p-value <0.001 in the population. The methods also comprise comparing the first SNP genotype to a corresponding first reference SNP genotype and predicting palm oil yield of the test plant based on extent of matching of the SNP genotypes.

### TECHNICAL FIELD

This application relates to methods for predicting palm oil yield of a test oil palm plant, and more particularly to methods for predicting palm oil yield of a test oil palm plant comprising determining, from a sample of a test oil palm plant of a population of oil palm plants, at least a first single nucleotide polymorphism (SNP) genotype of the test oil palm plant, the first SNP genotype corresponding to a first SNP marker, comparing the first SNP genotype of the test oil palm plant to a corresponding first reference SNP genotype indicative of the high-oil-production trait in the same genetic background as the population, and predicting palm oil yield of the test oil palm plant based on the extent to which the first SNP genotype of the test oil palm plant matches the corresponding first reference SNP genotype.

### BACKGROUND ART

The African oil palm Elaeis guineensis Jacq. is an important oil-food crop. Oil palm plants are monoecious, i.e. single plants produce both male and female flowers, and are characterized by alternating series of male and female inflorescences. The male inflorescence is made up of numerous spikelets, and can bear well over 100,000 flowers. Oil palm is naturally cross-pollinated by insects and wind. The female inflorescence is a spadix which contains several thousands of flowers borne on thorny spikelets. A bunch carries 500 to 4,000 fruits. The oil palm fruit is a sessile drupe that is spherical to ovoid or elongated in shape and is composed of an exocarp, a mesocarp containing palm oil, and an endocarp surrounding a kernel.

Oil palm is important both because of its high yield and because of the high quality of its oil. Regarding yield, oil palm is the highest yielding oil-food crop, with a recent average yield of 3.67 tonnes per hectare per year and with best progenies known to produce about 10 tonnes per hectare per year. Oil palm is also the most efficient plant known for harnessing the energy of sunlight for producing oil. Regarding quality, oil palm is cultivated for both palm oil, which is produced in the mesocarp, and palm kernel oil, which is produced in the kernel. Palm oil in particular is a balanced oil, having almost equal proportions of saturated fatty acids (≈55% including 45% of palmitic acid) and unsaturated fatty acids (≈45%), and it includes beta carotene. The palm kernel oil is more saturated than the mesocarp oil. Both are low in free fatty acids. The current combined output of palm oil and palm kernel oil is about 50 million tonnes per year, and demand is expected to increase substantially in the future with increasing global population and per capita consumption of oils and fats.

Although oil palm is the highest yielding oil-food crop, current oil palm crops produce well below their theoretical maximum, suggesting potential for improving yields of palm oil through improved selection and identification of high yielding oil palm plants. Conventional methods for identifying potential high-yielding palms, for use in crosses to generate progeny with higher yields as well as for commercial production of palm oil, require cultivation of palms and measurement of production of oil thereby over the course of many years, though, which is both time and labor intensive. Moreover, the conventional methods are based on direct measurement of oil content of sampled fruits, and thus result in destruction of the sampled fruits. In addition, conventional breeding techniques for propagation of oil palm for oil production are also time and labor intensive, particularly because the most productive, and thus commercially relevant, palms exhibit a hybrid phenotype which makes propagation thereof by direct hybrid crosses impractical. Quantitative trait loci (also termed QTL) marker programs based on linkage analysis have been implemented in oil palm with the aim of improving upon conventional breeding techniques, as taught for example by Billotte et al., Theoretical & Applied Genetics 120:1673-1687 (2010). Linkage analysis is based on recombination observed in a family within recent generations and often identifies poorly localized QTLs for complex phenotypes, though, and thus large families are needed for better detection and confirmation of QTLs, limiting practicality of this approach for oil palm. QTL marker programs based on association analysis for the purpose of identifying candidate genes may be a possibility for oil palm too, as discussed for example by Ong et. al, WO2014/129885, with respect to plant height. A primary focus on identifying candidate genes may be of limited benefit in the context of traits that are determined by multiple genes though, particularly genes that exhibit low penetrance with respect to the trait. QTL marker programs based on genome-wide association studies have been carried out in human and rice, among others, as taught by Hirota et al., Nature Genetics 44:1222-1226 (2012), and Huang et al., Nature Genetics 42:961-967 (2010), respectively. Application of this approach to oil palm has not been practical, though, because commercial palms tend to be generated from genetically narrow breeding materials. Accordingly, a need exists to improve oil palm through improved methods for predicting palm oil yields of oil palm plants.

### DISCLOSURE OF INVENTION

In one example embodiment, a method for predicting palm oil yield of a test oil palm plant is disclosed. The method comprises a step of (i) determining, from a sample of a test oil palm plant of a population of oil palm plants, at least a first single nucleotide polymorphism (SNP) genotype of the test oil palm plant. The first SNP genotype corresponds to a first SNP marker. The first SNP marker is located in a first candidate gene region for a high-oil-production trait. The first SNP marker also is associated, after stratification and kinship correction, with the high-oil-production trait with a p-value <0.001 in the population or has a linkage disequilibrium r² value of at least 0.2 with respect to a first other SNP marker that is linked thereto and associated, after stratification and kinship correction, with the high-oil-production trait a p-value <0.001 in the population. The method also comprises a step of (ii) comparing the first SNP genotype of the test oil palm plant to a corresponding first reference SNP genotype indicative of the high-oil-production trait in the same genetic background as the population. The method also comprises a step of (iii) predicting palm oil yield of the test oil palm plant based on the extent to which the first SNP genotype of the test oil palm plant matches the corresponding first reference SNP genotype. The first candidate gene region is a region of the oil palm genome corresponding to:

(1) candidate gene region 1, comprising a gene encoding CBL-Interacting Protein Kinase 32 and extending from 4 kb upstream to 4 kb downstream of the gene encoding CBL-Interacting Protein Kinase 32;

(2) candidate gene region 2, comprising a gene encoding Shaggy-Related Protein Kinase and extending from 4 kb upstream to 4 kb downstream of the gene encoding Shaggy-Related Protein Kinase;

(3) candidate gene region 3, comprising a gene encoding Probable Receptor-Like Protein Kinase and extending from 4 kb upstream to 4 kb downstream of the gene encoding Probable Receptor-Like Protein Kinase;

(4) candidate gene region 4, comprising a gene encoding Tau Class Glutathione S-Transferase and extending from 4 kb upstream to 4 kb downstream of the gene encoding Tau Class Glutathione S-Transferase; or

(5) candidate gene region 5, comprising a gene encoding Cinnamoyl-CoA Reductase and extending from 4 kb upstream to 4 kb downstream of the gene encoding Cinnamoyl-CoA Reductase.

In another example embodiment, a method for predicting palm oil yield of a test oil palm plant is disclosed. The method comprises a step of (i) determining, from a sample of a test oil palm plant of a population of oil palm plants, at least a first single nucleotide polymorphism (SNP) genotype of the test oil palm plant. The first SNP genotype corresponds to a first SNP marker. The first SNP marker is located in a first candidate gene region for a high-oil-production trait. The first SNP marker also is associated, after stratification and kinship correction, with the high-oil-production trait with a p-value <0.001 in the population or has a linkage disequilibrium r² value of at least 0.2 with respect to a first other SNP marker that is linked thereto and associated, after stratification and kinship correction, with the high-oil-production trait a p-value <0.001 in the population. The method also comprises a step of (ii) comparing the first SNP genotype of the test oil palm plant to a corresponding first reference SNP genotype indicative of the high-oil-production trait in the same genetic background as the population. The method also comprises a step of (iii) predicting palm oil yield of the test oil palm plant based on the extent to which the first SNP genotype of the test oil palm plant matches the corresponding first reference SNP genotype. The first candidate gene region is a region of the oil palm genome corresponding to:

(1) candidate gene region 6, comprising a gene encoding 1-Aminocyclopropane-1-Carboxylate Synthase 7 and extending from 4 kb upstream to 4 kb downstream of the gene encoding 1-Aminocyclopropane-1-Carboxylate Synthase 7;

(2) candidate gene region 7, comprising a gene encoding Mitochondrial Trans-2-Enoyl-CoA Reductase and extending from 4 kb upstream to 4 kb downstream of the gene encoding Mitochondrial Trans-2-Enoyl-CoA Reductase; or

(3) candidate gene region 8, comprising a gene encoding Chloroplastic Biotin Carboxyl Carrier Protein of Acetyl CoA Carboxylase and extending from 4 kb upstream to 4 kb downstream of the gene encoding Chloroplastic Biotin Carboxyl Carrier Protein of Acetyl CoA Carboxylase.

### BRIEF DESCRIPTION OF DRAWINGS

FIG. 1 shows quartile-quartile (Q-Q) plots of observed −log₁₀(p-values) versus expected −log₁₀(p-values) for genome-wide association studies (also termed GWAS) based on a naive model in (a) a Deli dura x AVROS pisifera population and (b) a Nigerian dura x AVROS pisifera population.

FIG. 2 shows (a, b) Q-Q plots of observed −log₁₀(p-values) versus expected −log₁₀(p-values) for GWAS and (c, d) Manhattan plots, all based on a compressed mixed linear model (also termed MLM), in (a, c) a Deli dura x AVROS pisifera population and (b, d) a Nigerian dura x AVROS pisifera population.

FIG. 3 is a diagram of candidate gene region 1, including an 11 kb predicted transcription unit, designated SDt092051, corresponding to CBL-Interacting Protein Kinase 32, including 18 predicted exons and 15 SNP markers, and a corresponding linkage disequilibrium block.

FIG. 4 shows box plots of oil-to-dry mesocarp for palms grouped according to SNP marker S_00002156_00008396 genotypes AA, AG, and GG, for (A) the AVROS cluster, (B) the UR_ALL cluster, and (C) the UR x AVROS cluster. See Examples for p-values.

FIG. 5 shows (A) a graph of relative expression of the SDt092051 transcript for eight high-yielding palms and eight low-yielding palms at weeks 12, 14, 16, 18, 20, and 22 after anthesis, grouped according to SNP marker S_00002156_00008396 genotypes AA, AG, and GG, (B) a graph of relative expression of the SDt092051 transcript for eight high-yielding palms and eight low-yielding palms at weeks 12, 14, 16, 18, 20, and 22 after anthesis, grouped according to low-yielding palms and high-yielding palms, and (C) mean oil-to-dry mesocarp for the eight high-yielding palms and the eight low-yielding palms, grouped according to SNP marker S_00002156_00008396 genotypes AA and presence of G (i.e. AG and GG).

FIG. 6 is a diagram of candidate gene region 2, including a predicted transcription unit, designated SDt093033, corresponding to Shaggy-Related Protein Kinase, including 11 predicted exons and 17 SNP markers, and corresponding linkage disequilibrium blocks.

FIG. 7 shows box plots of oil-to-dry mesocarp for palms grouped according to SNP marker S_000023169_00001770 genotypes AA, AG, and GG, for (A) the UR_ALL cluster, and (B) the UR x Dumpy AVROS cluster. See Examples for p-values.

FIG. 8 shows box plots of oil-to-dry mesocarp for palms grouped according to SNP marker S_000023169_00004772 genotypes AA, AC, and CC, for (A) the UR_ALL cluster, and (B) the UR x Dumpy AVROS cluster. See Examples for p-values.

FIG. 9 is a graph of relative expression of the SDt093033 transcript for eight high-yielding palms and eight low-yielding palms at weeks 12, 14, 16, 18, 20, and 22 after anthesis.

FIG. 10 shows mean oil-to-dry mesocarp for eight high-yielding palms and eight low-yielding palms, (A) grouped according to SNP marker S_000023169_00001770 genotypes AA, AG, and GG, and (B) grouped according to SNP marker S_000023169_00004772 genotypes AA, AC, and CC.

FIG. 11 is a diagram of candidate gene region 3, including a predicted transcription unit, designated SDt026153, corresponding to Probable Receptor-Like Protein Kinase, including 5 predicted exons and 21 SNP markers, and corresponding linkage disequilibrium blocks.

FIG. 12 shows box plots of oil-to-dry mesocarp for palms of the Dumpy AVROS cluster grouped according to (A) SNP marker S_000004932_00093119 genotypes AA, AG, and GG, and (B) SNP marker S_000004932_00094229 genotypes AA, AG, and GG. See Examples for p-values.

FIG. 13 shows box plots of oil-to-dry mesocarp for palms of the Dumpy AVROS cluster grouped according to (A) SNP marker S_000004932_00094970 genotypes AA, AG, and GG, and (B) SNP marker S_000004932_00097689 genotypes AA, AC, and CC. See Examples for p-values.

FIG. 14 shows box plots of oil-to-dry mesocarp for palms of the AVROS cluster grouped according to (A) SNP marker S_000004932_00093119 genotypes AA, AG, and GG, (B) SNP marker S_000004932_00094229 genotypes AA, AG, and GG, (C) SNP marker S_000004932_00094970 genotypes AA, AG, and GG, and (D) SNP marker S_000004932_00097689 genotypes AA, AC, and CC. See Examples for p-values.

FIG. 15 is a diagram of candidate gene region 4, including a predicted transcription unit, designated SDt081517, corresponding to Tau Class Glutathione S-Transferase, including 2 predicted exons and 7 SNP markers, and a corresponding linkage disequilibrium block.

FIG. 16 shows box plots of oil-to-dry mesocarp for palms grouped according to SNP marker S_00004607_00009651 genotypes AA and AG, for (A) the UR x AVROS cluster, and (B) the AVROS cluster. See Examples for p-values.

FIG. 17 is a diagram of candidate gene region 5, including a predicted transcription unit, designated SDt076624, corresponding to Cinnamoyl-CoA Reductase, including 6 predicted exons and 15 SNP markers, and corresponding linkage disequilibrium blocks.

FIG. 18 shows box plots of oil-to-dry mesocarp for palms grouped according to SNP marker S_00007694_00038606 genotypes AA, AG, and GG, for (A) the AVROS cluster, and (B) the Johor Labis x AVROS cluster. See Examples for p-values.

FIG. 19 shows box plots of oil-to-dry mesocarp for palms grouped according to SNP marker S_00007694_00038606 genotypes AA, AG, and GG, for (A) the UR_ALL cluster, and (B) the UR x Dumpy AVROS cluster. See Examples for p-values.

FIG. 20 shows box plots of oil-to-dry mesocarp for palms grouped according to SNP marker S_00007694_00038606 genotypes AA, AG, and GG, for the UR x AVROS cluster. See Examples for p-values.

FIG. 21 is a diagram of candidate gene region 6, including a predicted transcription unit, designated SDt076123, corresponding to 1-Aminocyclopropane-1-Carboxylate Synthase 7, including 4 predicted exons and 10 SNP markers, and corresponding linkage disequilibrium blocks.

FIG. 22 shows box plots of oil-to-dry mesocarp for palms grouped according to SNP marker S_00000302_00166120 genotypes AA, AG, and GG, for the Dumpy AVROS cluster. See Examples for p-values.

FIG. 23 shows box plots of oil-to-dry mesocarp for palms grouped according to SNP marker S_00000302_00166120 genotypes AA, AG, and GG, for (A) the AVROS cluster, and (B) the Johor Labis x AVROS cluster. See Examples for p-values.

FIG. 24 is a diagram of candidate gene region 7, including a predicted transcription unit, designated SDt098109, corresponding to Mitochondrial Trans-2-Enoyl-CoA Reductase, including 11 predicted exons and 25 SNP markers, and corresponding linkage disequilibrium blocks.

FIG. 25 shows box plots of oil-to-dry mesocarp for palms grouped according to SNP marker S_00002174_00010170 genotypes AA, AG, and GG, for the Dumpy AVROS cluster. See Examples for p-values.

FIG. 26 shows box plots of oil-to-dry mesocarp for palms grouped according to SNP marker S_00002174_00010170 genotypes AA, AG, and GG, for (A) the UR_ALL cluster and (B) the UR x AVROS cluster. See Examples for p-values.

FIG. 27 shows (A) a graph of relative expression of the SDt098109 transcript for eight high-yielding palms and eight low-yielding palms at weeks 12, 14, 16, 18, 20, and 22 after anthesis, grouped according to low-yielding palms and high-yielding palms, and (B) mean oil-to-dry mesocarp for the eight high-yielding palms and the eight low-yielding palms, grouped according to SNP marker S_00002174_00010170 genotypes AA and presence of G (i.e. AG and GG).

FIG. 28 is a diagram of candidate gene region 8, including a predicted transcription unit, designated SDt83215, corresponding to Chloroplastic Biotin Carboxyl Carrier Protein of Acetyl CoA Carboxylase, including 9 predicted exons and 15 SNP markers, and a corresponding linkage disequilibrium block.

FIG. 29 shows box plots of oil-to-dry mesocarp for palms of the AVROS cluster grouped according to (A) SNP marker S_00018257_00003287 genotypes AA, AC, and CC, and (B) SNP marker S_00018257_00006313 genotypes AA, AG, and GG. See Examples for p-values.

FIG. 30 shows box plots of oil-to-dry mesocarp for palms of the UR x AVROS cluster grouped according to (A) SNP marker S_00018257_00003287 genotypes AA, AC, and CC, and (B) SNP marker S_00018257 _00006313 genotypes AA, AG, and GG. See Examples for p-values.

### BEST MODE FOR CARRYING OUT THE INVENTION

The application is drawn to methods for predicting palm oil yield of a test oil palm plant. The methods comprise steps of (i) determining, from a sample of a test oil palm plant of a population of oil palm plants, at least a first single nucleotide polymorphism (SNP) genotype of the test oil palm plant, (ii) comparing the first SNP genotype of the test oil palm plant to a corresponding first reference SNP genotype indicative of the high-oil-production trait in the same genetic background as the population, and (iii) predicting palm oil yield of the test oil palm plant based on the extent to which the first SNP genotype of the test oil palm plant matches the corresponding first reference SNP genotype. The first SNP genotype corresponds to a first SNP marker. The first SNP marker is located in a first candidate gene region for a high-oil-production trait. The first SNP marker also is associated, after stratification and kinship correction, with the high-oil-production trait with a p-value <0.001 in the population or has a linkage disequilibrium r² value of at least 0.2 with respect to a first other SNP marker that is linked thereto and associated, after stratification and kinship correction, with the high-oil-production trait a p-value <0.001 in the population. In one example embodiment, the first candidate gene region is a region of the oil palm genome corresponding to one of candidate gene regions 1 to 5, as described in more detail below. In another example embodiment, the first candidate gene region is a region of the oil palm genome corresponding to one of candidate gene regions 6 to 8, as also described in more detail below.

By carrying out a combination of hypothesis-free genome-wide association studies, including stratification and kinship correction, for identification of SNP markers associated with a high-oil-production-trait, and hypothesis-driven candidate gene region studies, based on transcriptomics, proteomics, and metabolomics, for identification of candidate gene regions within the oil palm genome, it has been determined that SNP markers that are located in candidate gene regions 1-5 and candidate gene regions 6-8 of the oil palm genome and that are associated, after stratification and kinship correction, with a high-oil-production trait can be used to predict palm oil yield of test oil palm plants. Without wishing to be bound by theory, it is believed that the combination of the hypothesis-free genome-wide association studies and the hypothesis-driven candidate gene region studies revealed SNP markers for predicting palm oil yields, and corresponding candidate gene regions that contribute in determining palm oil yields, that would not have been apparent from either genome-wide association studies or candidate gene region studies alone. In this regard, it is believed that stratification and kinship correction reduce false-positive signals due to recent common ancestry of small groups of individuals within the population of oil palm plants from which a test oil palm plant is sampled. Moreover, it is believed that focusing on SNPs that are located within candidate gene regions allows for relaxation of statistical criteria for identification of SNPs as useful for predicting oil palm yields. In addition, it is believed that the combination of genome-wide association studies and candidate gene region studies provide a basis for identifying consistency, coherency, and biological response with respect to SNP markers and candidate gene regions. Accordingly, the combination allows for identification of SNPs that are useful for predicting palm oil yields that would otherwise have been missed.

As noted, the first candidate gene region can be a region of the oil palm genome corresponding to: (1) candidate gene region 1, comprising a gene encoding CBL-Interacting Protein Kinase 32 and extending from 4 kb upstream to 4. kb downstream of the gene encoding CBL-Interacting Protein Kinase 32; (2) candidate gene region 2, comprising a gene encoding Shaggy-Related Protein Kinase and extending from 4 kb upstream to 4 kb downstream of the gene encoding Shaggy-Related Protein Kinase; (3) candidate gene region 3, comprising a gene encoding Probable Receptor-Like Protein Kinase and extending from 4 kb upstream to 4 kb downstream of the gene encoding Probable Receptor-Like Protein Kinase; (4) candidate gene region 4, comprising a gene encoding Tau Class Glutathione S-Transferase and extending from 4 kb upstream to 4 kb downstream of the gene encoding Tau Class Glutathione S-Transferase; or (5) candidate gene region 5, comprising a gene encoding Cinnamoyl-CoA Reductase and extending from 4 kb upstream to 4 kb downstream of the gene encoding Cinnamoyl-CoA Reductase. Without wishing to be bound by theory, it is hypothesized that CBL-Interacting Protein Kinase 32, Shaggy-Related Protein Kinase, and Probable Receptor-Like Protein Kinase may influence oil biosynthesis pathway genes via phosphorylation, that Tau Class Glutathione S-Transferase may be a useful marker for oil yield, expressed in terms of oil-to-dry mesocarp, as mediated via micro-RNA, and that Cinnamoyl-CoA Reductase can influence oil yield, again expressed in terms of oil-to-dry mesocarp, by influencing lignin content and architecture of fruit as mediated via splicing activity.

As also noted the first candidate gene region also can be a region of the oil palm genome corresponding to: (1) candidate gene region 6, comprising a gene encoding 1-Aminocyclopropane-1-Carboxylate Synthase 7 and extending from 4 kb upstream to 4 kb downstream of the gene encoding 1-Aminocyclopropane-1-Carboxylate Synthase 7; (2) candidate gene region 7, comprising a gene encoding Mitochondrial Trans-2-Enoyl-CoA Reductase and extending from 4 kb upstream to 4 kb downstream of the gene encoding Mitochondrial Trans-2-Enoyl-CoA Reductase; or (3) candidate gene region 8, comprising a gene encoding Chloroplastic Biotin Carboxyl Carrier Protein of Acetyl CoA Carboxylase and extending from 4 kb upstream to 4 kb downstream of the gene encoding Chloroplastic Biotin Carboxyl Carrier Protein of Acetyl CoA Carboxylase. Without wishing to be bound by theory, it is hypothesized that 1-Aminocyclopropane-1-Carboxylate Synthase 7 may be a useful marker for oil yield, expressed in terms of oil-to-dry mesocarp, as mediated via promoter activity, that Mitochondrial Trans-2-Enoyl-CoA Reductase may serve as a marker for high yielding palms, and that Chloroplastic Biotin Carboxyl Carrier Protein of Acetyl CoA Carboxylase may be a useful marker for oil yield, again expressed in terms of oil-to-dry mesocarp, as mediated via its promoter binding site.

The methods will enable identification of potential high-yielding palms, for use in crosses to generate progeny with higher yields and for commercial production of palm oil, without need for cultivation of the palms to maturity, thus bypassing the need for the time and labor intensive cultivations and measurements, the destructive sampling of fruits, and the impracticality of direct hybrid crosses that are characteristic of conventional approaches. For example, the methods can be used to choose oil palm plants for germination, cultivation in a nursery, cultivation for commercial production of palm oil, cultivation for further propagation, etc., well before direct measurement of palm oil production by the test oil palm plant could be accomplished. Also for example, the methods can be used to accomplish prediction of palm oil yields with greater efficiency and/or less variability than by direct measurement of palm oil production. The methods can be used advantageously with respect to even a single SNP, given that improvements in oil palm yield that seem small on a percentage basis still can have a dramatic effect on overall palm oil yields, given the large scale of commercial cultivations. The methods also can be used advantageously with respect to combinations of two or more SNPs, e.g. a first SNP genotype of a first SNP marker located in a first candidate gene region, and a second SNP genotype of a second SNP marker of a second candidate gene region, given additive and/or synergistic effects.

The terms “high-oil-production trait,” “high yield,” “high-yielding,” and “oil yield,” as used with respect to the methods disclosed herein, refer to yields of palm oil in mesocarp tissue of fruits of oil palm plants.

The singular forms “a,” “an,” and “the” are intended to include the plural forms as well, unless the context clearly indicates otherwise.

As noted above, in one example embodiment a method for predicting palm oil yield of a test oil palm plant is disclosed. The method comprises a step of (i) determining, from a sample of a test oil palm plant of a population of oil palm plants, at least a first single nucleotide polymorphism (also termed SNP) genotype of the test oil palm plant.

The SNP genotype of the test oil palm plant corresponds to the constitution of SNP alleles at a particular locus, or position, on each chromosome in which the locus occurs in the genome of the test oil palm plant. A SNP is a polymorphic variation with respect to a single nucleotide that occurs at such a locus on a chromosome. A SNP allele is the specific nucleotide present at the locus on the chromosome. For oil palm plants, which are diploid and which thus inherit one set of maternally derived chromosomes and one set of paternally derived chromosomes, the SNP genotype corresponds to two SNP alleles, one at the particular locus on the maternally derived chromosome and the other at the particular locus on the paternally derived chromosome. Each SNP allele may be classified, for example, based on allele frequency, e.g. as a major allele (A) or a minor allele (a). Thus, for example, the SNP genotype can correspond to two major alleles (A/A), one major allele and one minor allele (A/a), or two minor alleles (a/a). A SNP allele also may be classified, for example, based on the nucleotide that constitutes the SNP allele, e.g. G, A, T, or C.

The test oil palm plant can be an oil palm plant in any suitable form. For example, the test oil palm plant can be a seed, a seedling, a nursery phase plant, an immature phase plant, a cell culture plant, a zygotic embryo culture plant, or a somatic tissue culture plant. Also for example, the test oil palm plant can be a production phase plant, a mature palm, a mature mother palm, or a mature pollen donor.

A test oil palm plant in the form of a seed, a seedling, a nursery phase plant, an immature phase plant, a cell culture plant, a zygotic embryo culture plant, or a somatic tissue culture plant is in a form that is not yet mature, and thus that is not yet producing palm oil in amounts typical of commercial production, if at all. Accordingly, the method as applied to a test oil palm plant in such a form can be used to predict palm oil yield of the test oil palm plant before the test oil palm plant has matured sufficiently to allow direct measurement of palm oil production by the test oil palm plant during commercial production.

A test oil palm plant in the form of a production phase plant, a mature palm, a mature mother palm, or a mature pollen donor is in a form that is mature. Accordingly, the method as applied to a test oil palm plant in such a form can be used to predict palm oil yield of the test oil palm as an alternative to direct measurement of oil palm yield.

The population of oil palm plants from which the test oil palm plant is sampled can comprise any suitable population of oil palm plants. The population can be specified in terms of fruit type and/or identity of the breeding material from which the population was generated.

In this regard, fruit type is a monogenic trait in oil palm that is important with respect to breeding and commercial production. Oil palms with either of two distinct fruit types are generally used in breeding and seed production through crossing in order to generate palms for commercial production of palm oil, also termed commercial planting materials or agricultural production plants. The first fruit type is dura (genotype: sh+ sh+), which is characterized by a thick shell corresponding to 28 to 35% of the fruit by weight, with no ring of black fibres around the kernel of the fruit. For dura fruits, the ratio of mesocarp to fruit varies from 50 to 60%, with extractable oil content in proportion to bunch weight of 18 to 24%. The second fruit type is pisifera (genotype: sh− sh−), which is characterized by the absence of a shell, the vestiges of which are represented by a ring of fibres around a small kernel. Accordingly, for pisifera fruits, the ratio of mesocarp to fruit is 90 to 100%. The ratio of mesocarp oil to bunch is comparable to the dura at 16 to 28%. Pisiferas are however usually female sterile as the majority of bunches abort at an early stage of development.

Crossing dura and pisifera gives rise to palms with a third fruit type, the tenera (genotype: sh+ sh−). Tenera fruits have thin shells of 8 to 10% of the fruit by weight, corresponding to a thickness of 0.5 to 4 mm, around which is a characteristic ring of black fibres. For tenera fruits, the ratio of mesocarp to fruit is comparatively high, in the range of 60 to 80%. Commercial tenera palms generally produce more fruit bunches than duras, although mean bunch weight is lower. The ratio of mesocarp oil to bunch is in the range of 20 to 30%, the highest of the three fruit types, and thus tenera are typically used as commercial planting materials.

Identity of the breeding material can be based on the source and breeding history of the breeding material. Dura palm breeding populations used in Southeast Asia include Serdang Avenue, Ulu Remis (which incorporated some Serdang Avenue material), Johor Labis, and Elmina estate, including Deli Dumpy, all of which are derived from Deli dura. Pisifera breeding populations used for seed production are generally grouped as Yangambi, AVROS, Binga and URT. Other dura and pisifera populations are used in Africa and South America.

Oil palm breeding is primarily aimed at selecting for improved parental dura and pisifera breeding stock palms for production of superior tenera commercial planting materials. Such materials are largely in the form of seeds although the use of tissue culture for propagation of clones continues to be developed. Generally, parental dura breeding populations are generated by crossing among selected dura palms. Based on the monogenic inheritance of fruit type, 100% of the resulting palms will be duras. After several years of yield recording and confirmation of bunch and fruit characteristics, duras are selected for breeding based on phenotype. In contrast, pisifera palms are normally female sterile and thus breeding populations thereof must be generated by crossing among selected teneras or by crossing selected teneras with selected pisiferas. The tenera x tenera cross will generate 25% duras, 50% teneras and 25% pisiferas. The tenera x pisifera cross will generate 50% teneras and 50% pisiferas. The yield potential of pisiferas is then determined indirectly by progeny testing with the elite duras, i.e. by crossing duras and pisiferas to generate teneras, and then determining yield phenotypes of the fruits of the teneras over time. From this, pisiferas with good general combining ability are selected based on the performance of their tenera progenies. Intercrossing among selected parents is also carried out with progenies being carried forward to the next breeding cycle. This allows introduction of new genes into the breeding programme to increase genetic variability.

Oil palm cultivation for commercial production of palm oil can be improved by use of the superior tenera commercial planting materials. Priority selection objectives include high oil yield per unit area in terms of high fresh fruit bunch yield and high oil to bunch ratio (thin shell, thick mesocarp), high early yield (precocity), and good oil qualities, among other traits. Progeny plants may be cultivated by conventional approaches, e.g. seedlings may be cultivated in polyethylene bags in pre-nursery and nursery settings, raised for about 12 months, and then planted as seedlings, with progeny that are known or predicted to exhibit high yields chosen for further cultivation, among other approaches.

Accordingly, in some examples the population of oil palm plants can comprise a Ulu Remis dura x AVROS pisifera population, a Johor Labis dura x AVROS pisifera population, a Ulu Remis dura x Dumpy AVROS pisifera population, a Dumpy AVROS population, or a combination thereof. Also in some examples the population of oil palm plants comprises a Ulu Remis dura x Ulu Remis dura population, a Ulu Remis dura x Johor Labis dura population, a Johor Labis dura x Johor Labis dura population, an AVROS pisifera x AVROS tenera population, an AVROS tenera x AVROS tenera population, a Dumpy AVROS pisifera x Dumpy AVROS tenera population, a Dumpy AVROS tenera x Dumpy AVROS tenera population, or a combination thereof.

The population of oil palm plants can be characterized in terms of one or more clusters of oil palm plants based on genetic background of the oil palm plants of the population. The clusters can correspond to distinct populations, e.g. as two clusters that do not overlap with each other, or can correspond to overlapping populations, e.g. as two clusters that partially or completely overlap with each other. For example, the population may be characterized as a UR x AVROS cluster, a Johor Labis x AVROS cluster, a UR x Dumpy AVROS cluster, a Dumpy AVROS cluster, an AVROS cluster, a UR_ALL cluster, and a Nigerian dura x AVROS pisifera cluster. For reference, the UR x AVROS cluster is a cluster of individual palms made up of Ulu Remis dura and AVROS pisifera. The Johor Labis x AVROS cluster is a cluster of individual palms made up of Johor Labis dura and AVROS pisifera. The UR x Dumpy AVROS cluster is a cluster of individual palms made up of Ulu Remis dura and Dumpy AVROS pisifera. The AVROS cluster refers to a cluster of individual palms made up of AVROS dura and Johor Labis dura, which means that these individual share a similar paternal line, AVROS. The UR_ALL cluster refers to all individual palms with Ulu Remis dura and AVROS pisifera or Dumpy AVROS pisifera, such that all individuals in this cluster share the maternal line Ulu Remis dura.

The sample of the test oil palm plant can comprise any organ, tissue, cell, or other part of the test oil palm plant that includes sufficient genomic DNA of the test oil palm plant to allow for determination of one or more SNP genotypes of the test oil palm plant, e.g. the first SNP genotype. For example, the sample can comprise a leaf tissue, among other organs, tissues, cells, or other parts. As one of ordinary skill will appreciate, determining, from a sample of a test oil palm plant, one or more SNP genotypes of the test oil palm plant, is necessarily transformative of the sample. The one or more SNP genotypes cannot be determined, for example, merely based on appearance of the sample. Rather, determination of the one or more SNP genotypes of the test oil palm plant requires separation of the sample from the test oil palm plant and/or separation of genomic DNA from the sample.

Determination of the at least first SNP genotype can be carried out by any suitable technique, including, for example, whole genome resequencing with SNP calling, hybridization-based methods, enzyme-based methods, or other post-amplification methods, among others.

The first SNP genotype corresponds to a first SNP marker. A SNP marker is a SNP that can be used in genetic mapping.

The first SNP marker is located in a first candidate gene region for a high-oil-production trait. A candidate gene region, as the term is used herein, is a locus, extending along a portion of a chromosome, that (1) includes a gene, corresponding to a region of DNA that is a transcription unit, bounded by an initiation site and a termination site, that is transcribed into a primary transcript, for production of a protein or RNA, (2) extends from 4 kb upstream of the gene to 4 kb downstream of the gene, and (3) contributes in determining a phenotype of the test oil palm plant, i.e. in this case, the high-oil-production trait.

The high-oil-production trait relates to a trait of production of palm oil by the test oil palm plant upon reaching a mature state, e.g. reaching production phase, and upon being cultivated under conditions suitable for production of palm oil in a high amount, e.g. commercial cultivation, in an amount that is higher than average, with respect to the population of oil palm plants from which the test oil palm plant is sampled, also upon reaching a mature state and upon being cultivated under conditions suitable for production of palm oil in a high amount.

Considering a test oil palm plant that is a tenera oil palm plant, the high-oil-production trait can correspond, for example, to production of palm oil at greater than 3.67 tonnes of palm oil per hectare per year, i.e. above recent average yields for typical oil palm plants used in commercial production, which also are tenera oil palm plants, as discussed above. The high-oil production trait also can correspond, for example, to production of palm oil at greater than 10 tonnes of palm oil per hectare per year, i.e. above recent average yields for current best-progeny oil palm plants used in commercial production. The high-oil production trait also can correspond, for example, to production of palm oil at greater than 4, 5, 6, 7, 8, or 9 tonnes of palm oil per hectare per year, i.e. above yields that are intermediate between the recent average yields noted above. Considering a test oil palm plant that is a dura oil palm plant or a pisifera oil palm plant, the high-oil production trait can correspond to production of palm oil in correspondingly lower amounts, consistent with lower average yields obtained for dura and pisifera oil palm plants relative to tenera oil palm plants.

The high-oil-production trait can comprise increased oil-to-dry mesocarp (also termed O/DM or O_DM). As noted above, palm oil is produced in the mesocarp of the oil palm fruit. O/DM is a measure of palm oil yield. Accordingly, a relatively high O/DM is an indicator of relatively high production of palm oil.

The first SNP marker is associated, after stratification and kinship correction, with the high-oil-production trait with a p-value <0.001 in the population or has a linkage disequilibrium r² value of at least 0.2 with respect to a first other SNP marker that is linked thereto and associated, after stratification and kinship correction, with the high-oil-production trait with a p-value <0.001 in the population.

A first SNP marker being associated, after stratification and kinship correction, with a trait with a p-value <0.001 in the population indicates that the first SNP maker and the trait likely are linked.

A p-value is the probability of observing a test statistic, in this case relating to association of a SNP marker, e.g. the first SNP marker or the first other SNP marker, and the high-oil-production trait, equal to or greater than a test statistic actually observed, if the null hypothesis is true and thus there is no association, as discussed, for example, by Bush & Moore, Chapter 11: Genome-Wide Association Studies, PLOS Computational Biology 8(12):e1002822, 1-11 (2012). Accordingly, a p-value <0.001 in the population indicates that the likelihood that the observed test statistic, relating to association, would have been observed in the absence of association is low.

Stratification and kinship correction are taken into account in determining the association. As noted above, stratification and kinship correction reduce false-positive signals due to recent common ancestry of small groups of individuals within the population of oil palm plants from which the test oil palm plant is sampled, which, in combination with candidate gene regions studies, allows for identification of SNPs that are useful for predicting palm oil yields that would otherwise have been missed

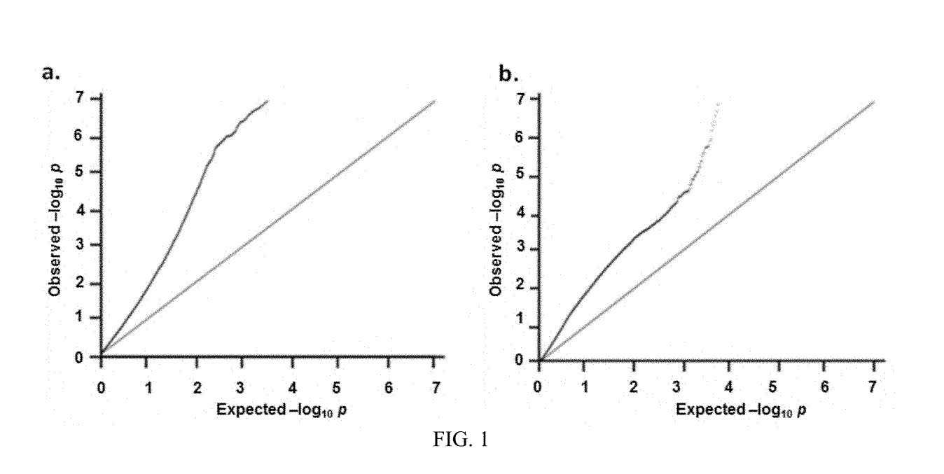

For purposes of illustration here, a genome-wide association study (also termed GWAS) was performed on a Deli x AVROS oil palm population and a Nigerian x AVROS oil palm population, respectively using a naive model. The method only measured the association between the markers and the trait of interest regardless of population structures, or families, of the mapping population. According to quartile-quartile (Q-Q) plots and genomic inflation factor (GIF) estimations, −log₁₀(p-values) that were heavily inflated were observed, specifically indicating 4017 and 24760 SNPs to be associated with O/DM. As shown in FIG. 1, Deli x AVROS with GIF=3.66 and Nigerian x AVROS with GIF=11.9 indicated early deviation of the observed −log₁₀(p-value) from the null expectation (y=x), respectively. For reference, O/DM phenotype data for the Deli x AVROS population were as follows: mean 76.87%; standard deviation 2.28; coefficient of variation 2.96; median 77.10%; and range 18.60%. O/DM phenotype data for the Nigerian x AVROS population were as follows: mean 75.67%; standard deviation 2.43; coefficient of variation 3.22; median 75.80%; and range 14.20%. Most of these indicated SNPs only explained origin effects, not trait variants, and thus were false-positive signals. The naive model failed to account for the recent common ancestry of small groups of individuals, defined as cryptic relatedness, in accordance with Astle & Balding, Statistical Science 24:451-471 (2009), here posing a more serious confounding problem than population structure to the GWAS, in accordance with Devlin & Roeder, Biometrics 55:997-1004 (1999).

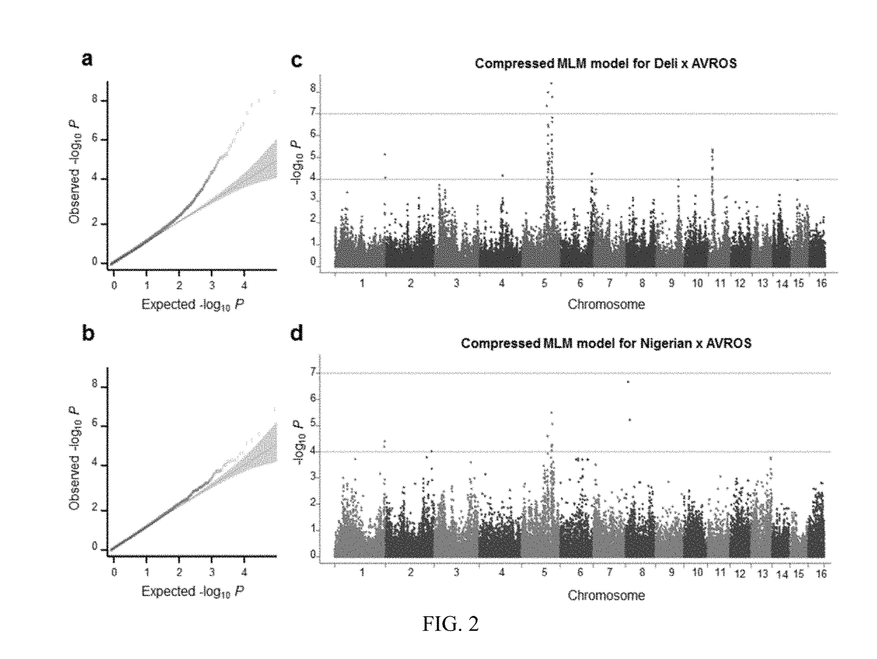

A subsequent GWAS based on a compressed mixed linear model (also termed MLM) with population parameters previously determined (P3D) was carried out toward addressing the problem of genomic inflations using principal component analysis and a group kinship matrix. This approach greatly reduced false positives, specifically resulting in 70 and 18 O/DM-associated SNPs in Deli x AVROS and Nigerian x AVROS, respectively. Specifically, as shown in FIG. 2, Q-Q plots in both populations showed that deviation of the observed statistics from the null expectation were delayed significantly. Moreover, the GIFs for Deli x AVROS and Nigerian x AVROS also declined to 1.1 and 1.9 (approaching an ideal GIF=1.0). The chromosomal distribution of the resulting SNPs for both populations can be visualized in Manhattan plots, as also shown in FIG. 2. Based on this approach, a total of 82 O/DM-associated SNPs were identified after excluding markers that overlapped in both populations.

Stratification and kinship correction can be applied similarly regarding other oil palm populations and/or clusters, e.g. the populations and clusters noted above.

Accordingly, for example, the first SNP marker being located in a first candidate gene region for a high-oil-production trait and being associated, after stratification and kinship correction, with the high-oil-production trait with a p-value <0.001 in the population can be a SNP marker for which association with the high-oil-production trait (i) has been confirmed based on a model that is not a naive model and/or (ii) would be confirmed based on a model that is not a naive model. Also for example, the first SNP marker being located in a first candidate gene region for a high-oil-production trait and being associated, after stratification and kinship correction, with the high-oil-production trait with a p-value <0.001 in the population can be a SNP marker for which association with the high-oil-production trait (i) has been confirmed based on a compressed mixed linear model with population parameters previously determined, carried out using principal component analysis and a group kinship matrix and/or (ii) would be confirmed based on a compressed mixed linear model with population parameters previously determined, carried out using principal component analysis and a group kinship matrix.

A first SNP marker having a linkage disequilibrium r² value of at least 0.2 with respect to a first other SNP marker that is linked thereto and associated, after stratification and kinship correction, with the high-oil-production trait with a p-value <0.001 in the population indicates the following. First, a high likelihood exists that an allele of the first SNP marker and an allele of the first other SNP marker are in linkage disequilibrium. Second, a high likelihood exists that the first other SNP marker and the trait are linked. In this regard, a linkage disequilibrium r² value relates to measuring likelihood that two loci are in linkage disequilibrium as an average pairwise correlation coefficient.

Accordingly, in some examples the first SNP marker associated, after stratification and kinship correction, with the high-oil-production trait with a p-value <0.001 in the population. Also, in some examples the first SNP marker has a linkage disequilibrium r² value of at least 0.2 with respect to a first other SNP marker that is linked thereto and associated, after stratification and kinship correction, with the high-oil-production trait with a p-value <0.001 in the population. Also, in some examples both apply.

The first candidate gene region can be a region of the oil palm genome corresponding to:

(1) candidate gene region 1, comprising a gene encoding CBL-Interacting Protein Kinase 32 and extending from 4 kb upstream to 4 kb downstream of the gene encoding CBL-Interacting Protein Kinase 32;

(2) candidate gene region 2, comprising a gene encoding Shaggy-Related Protein Kinase and extending from 4 kb upstream to 4 kb downstream of the gene encoding Shaggy-Related Protein Kinase;

(3) candidate gene region 3, comprising a gene encoding Probable Receptor-Like Protein Kinase and extending from 4 kb upstream to 4 kb downstream of the gene encoding Probable Receptor-Like Protein Kinase;

(4) candidate gene region 4, comprising a gene encoding Tau Class Glutathione S-Transferase and extending from 4 kb upstream to 4 kb downstream of the gene encoding Tau Class Glutathione S-Transferase; or

(5) candidate gene region 5, comprising a gene encoding Cinnamoyl-CoA Reductase and extending from 4 kb upstream to 4 kb downstream of the gene encoding Cinnamoyl-CoA Reductase.

Considering candidate gene regions 1 to 5 in more detail, candidate gene region 1 includes an 11 kb predicted transcription unit that includes 18 predicted exons, corresponding to the gene encoding CBL-Interacting Protein Kinase 32, and extends from 4 kb upstream of the transcription unit to 4 kb downstream of the transcription unit. CBL-Interacting Protein Kinase 32 is a member of a calcium sensor protein family, the members of which interact with calcineurin B-like proteins, forming a complex and dynamic calcium-decoding signaling network, and are involved in abiotic stress regulation in maize, among other plants. Genome-wide association studies revealed that candidate gene region 1 includes 15 SNP markers, of which one SNP marker, designated S_00002156_00008396 and having a SNP genotype major allele AG and a SNP genotype minor allele AA, is associated, after stratification and kinship correction, with the high-oil-production trait with a p-value <0.001 in the population, with respect to the AVROS cluster, the UR_ALL cluster, and the UR x AVROS cluster. The S_00002156_00008396 SNP marker is located in candidate gene region 1 within the 5′ untranslated region of the gene encoding CBL-Interacting Protein Kinase 32, specifically 1861 base pairs from the ATG start codon thereof, and has a SNP change corresponding to “A>R (A/G).” Based on sequence analyses, it is predicted that a transcription factor binding site is provided overlapping the position of the S_00002156_00008396 SNP marker when the nucleotide at the SNP marker is A, and that no such transcription factor binding site is provided when the nucleotide at the SNP marker is G.

Candidate gene region 2 includes a predicted transcription unit that includes 11 predicted exons, corresponding to the gene encoding Shaggy-Related Protein Kinase, and extends from 4 kb upstream of the transcription unit to 4 kb downstream of the transcription unit. Shaggy-Related Protein Kinase is a member of Glycogen Synthase Kinase 3/SHAGGY-Like Kinase family, the members of which are involved in developmental processes of embryo, flower, and stomata, as well as wound response, and are involved in brassinosteroid signaling. Some members are upregulated transcriptionally in response to salt stress. Genome-wide association studies revealed that candidate gene region 2 includes 17 SNP markers, of which two SNP markers, the first designated S_000023169_00001770 and having a SNP genotype major allele AG and a SNP genotype minor allele GG, and the second designated S_000023169_00004772 and having a SNP genotype major allele AC and a SNP genotype minor allele AA, are associated, after stratification and kinship correction, with the high-oil-production trait with a p-value <0.001 in the population, with respect to the UR_ALL cluster and the UR x Dumpy AVROS cluster. The S_000023169_00001770 SNP marker is located in candidate gene region 2 within an intron of the gene encoding Shaggy-Related Protein Kinase, specifically 240 base pairs from exon 7, between exons 6 and 7, and has a SNP change corresponding to “A/G.” Sequence analysis indicates predicted splicing. The S_000023169_00004772 SNP marker is located in candidate gene region 2 within an intron of the gene encoding Shaggy-Related Protein Kinase, specifically 556 base pairs from exon 10, between exons 9 and 10, and has a SNP change corresponding to “A/C.” Sequence analysis indicates no predicted splicing.

Candidate gene region 3 includes a predicted transcription unit that includes 5 predicted exons, corresponding to the gene encoding Probable Receptor-Like Protein Kinase, and extends from 4 kb upstream of the transcription unit to 4 kb downstream of the transcription unit. Probable Receptor-Like Protein Kinase is a member of Receptor-Like Protein Kinase family, the members of which are involved in signal transduction of abscisic acid in Arabidopsis and response toward environmental stress. Genome-wide association studies revealed that candidate gene region 3 includes 21 SNP markers, of which four SNP markers, the first designated S_000004932_00093119 and having a SNP genotype major allele AG and a SNP genotype minor allele GG, the second designated S_000004932_00094229 and having a SNP genotype major allele AG and a SNP genotype minor allele AA, the third designated S_000004932_00094970 and having a SNP genotype major allele AG and a SNP genotype minor allele GG, and the fourth designated S_000004932_00097689 and having a SNP genotype major allele AC and a SNP genotype minor allele AA, are associated, after stratification and kinship correction, with the high-oil-production trait with a p-value <0.001 in the population, with respect to the Dumpy AVROS cluster. The S_000004932_00093119 SNP marker is located in candidate gene region 3 within the 3′-untranslated region of the gene encoding Probable Receptor-Like Protein Kinase, specifically 288 base pairs from exon 5, and has a SNP change corresponding to “A/G.” The S_000004932_00094229 SNP marker is located in candidate gene region 3 within an intron of the gene encoding Probable Receptor-Like Protein Kinase, specifically 277 base pairs from exon 5, between exons 4 and 5, and has a SNP change corresponding to “C/T.” The S_000004932_00094970 SNP marker is located in candidate gene region 3 within an intron of the gene encoding Probable Receptor-Like Protein Kinase, specifically 625 base pairs from exon 4, between exons 4 and 5, and has a SNP change corresponding to “T/C.” The S_000004932_00097689 SNP marker is located in candidate gene region 3 within an intron of the gene encoding Probable Receptor-Like Protein Kinase, specifically 673 base pairs from exon 3, between exons 2 and 3, and has a SNP change corresponding to “G/T.”

Candidate gene region 4 includes a predicted transcription unit that includes 2 predicted exons, corresponding to the gene encoding Tau Class Glutathione S-Transferase, and extends from 4 kb upstream of the transcription unit to 4 kb downstream of the transcription unit. Tau Class Glutathione S-Transferase is a member of the Glutathione S-Transferase family, the members of which catalyze conjugation of compounds to glutathione and target the compounds for storage in vacuoles or apoplasts. The compounds can include products of oxidative stress, carcinogens, and/or environmental toxins. Genome-wide association studies revealed that candidate gene region 4 includes 7 SNP markers, of which one SNP marker, designated S_00004607_00009651 and having a SNP genotype major allele AG and a SNP genotype minor allele AA, is associated, after stratification and kinship correction, with the high-oil-production trait with a p-value <0.001 in the population, with respect to the UR x AVROS cluster and the AVROS cluster. The S_00004607_00009651 SNP marker is located in candidate gene region 4 within the 3′ untranslated region of the gene encoding Tau Class Glutathione S-Transferase, specifically 3110 base pairs from the stop codon thereof, and has a SNP change corresponding to “A/G.”

Candidate gene region 5 includes a predicted transcription unit that includes 6 predicted exons, corresponding to the gene encoding Cinnamoyl-CoA Reductase, and extends from 4 kb upstream of the transcription unit to 4 kb downstream of the transcription unit. Cinnamoyl-CoA Reductase catalyzes the first specific step in the synthesis of monomers of lignin, specifically reaction of cinnamaldehyde, CoA, and NADP+ to cinnamoyl-CoA, NADPH, and H+. Genome-wide association studies revealed that candidate gene region 5 includes 15 SNP markers, of which one SNP marker, designated S_00007694_00038606 and having a SNP genotype major allele GG and a SNP genotype minor allele AA, is associated, after stratification and kinship correction, with the high-oil-production trait with a p-value <0.001 in the population, with respect to the AVROS cluster, the Johor Labis x AVROS cluster, the UR_ALL cluster, and the UR x AVROS cluster. The S_00007694_00038606 SNP marker is located in candidate gene region 5 within the 3′ untranslated region of the gene encoding Cinnamoyl-CoA Reductase, specifically 19 base pairs from the stop codon thereof, and has a SNP change corresponding to “A/G.” Sequence analysis indicates to miRNA binding site.

The method also comprises a step of (ii) comparing the first SNP genotype of the test oil palm plant to a corresponding first reference SNP genotype indicative of the high-oil-production trait in the same genetic background as the population.

The genetic background that is the same as the population can correspond, for example, to a population based on crossing oil palm plants of the same types as used to generate the population from which the test oil palm plant is sampled, e.g. a Ulu Remis dura x AVROS pisifera population, a Johor Labis dura x AVROS pisifera population, a Ulu Remis dura x Dumpy AVROS pisifera population, a Dumpy AVROS population, or a combination thereof, or a Ulu Remis dura x Ulu Remis dura population, a Ulu Remis dura x Johor Labis dura population, a Johor Labis dura x Johor Labis dura population, an AVROS pisifera x AVROS tenera population, an AVROS tenera x AVROS tenera population, a Dumpy AVROS pisifera x Dumpy AVROS tenera population, a Dumpy AVROS tenera x Dumpy AVROS tenera population, or a combination thereof. The genetic background that is the same as the population also can correspond, for example, to a population based on crossing the same individual oil palm plants used to generate the population from which the test oil palm plant is sampled. The genetic background that is the same as the population also can correspond, for example, to the same actual population from which the test oil palm plant is sampled.

The first reference SNP genotype indicative of the high-oil-production trait in the same genetic background as the population can correspond to the same SNP as the first SNP genotype, i.e. both can correspond to the same polymorphic variation with respect to a single nucleotide that occurs at a particular locus of a particular chromosome. The first reference SNP genotype can comprise one or more SNP alleles that, alone or together, indicate a higher likelihood that the test oil palm plant thereof exhibits, if mature, or will exhibit, upon reaching maturity, the high-oil-production trait, in comparison to oil palm plants of the same population that lack the one or more SNP alleles.

The method also comprises a step of (iii) predicting palm oil yield of the test oil palm plant based on the extent to which the first SNP genotype of the test oil palm plant matches the corresponding first reference SNP genotype. The first SNP genotype of the test oil palm plant can match the corresponding first reference SNP genotype based on both SNP genotypes sharing at least a first SNP allele indicative of the high-oil-production trait in the same genetic background as the population. In some examples the first SNP genotype and the first reference SNP genotype are heterozygous for the first allele indicative of the high-oil production trait, i.e. both have only one copy of the SNP allele. Also, in some examples the first SNP genotype and the first reference SNP genotype are homozygous for the first allele indicative of the high-oil production trait, i.e. both have two copies of the SNP allele. Also, in some examples the first SNP genotype is heterozygous for the first allele indicative of the high-oil production trait and the first reference SNP genotype is homozygous for the first allele indicative of the high-oil production trait. Also, in some examples the first SNP genotype is homozygous for the first allele indicative of the high-oil production trait and the first reference SNP genotype is heterozygous for the first allele indicative of the high-oil production trait.

The step of predicting palm oil yield of the test oil palm plant can further comprise applying a model, such as a genotype model, a dominant model, or a recessive model, among others, in order to facilitate the predicting. A genotype model tests the association of a trait, e.g. a high-oil production trait, with the presence of a SNP allele, either a major allele (A) or a minor allele (a). A dominant model tests the association of a trait, e.g. a high-oil production trait, with the presence of a SNP allele either as a homozygous genotype or a heterozygous genotype, e.g. the major allele either as a homozygous genotype (e.g. A/A) or a heterozygous genotype (e.g. A/a). A recessive model tests the association of a trait, e.g. a high-oil production trait, with the presence of a SNP allele as a homozygous genotype, e.g. the major allele as a homozygous genotype (A/A). Accordingly, in some examples, the predicting of palm oil yield of the test oil palm plant further comprises applying a genotype model. Also in some examples, the predicting of palm oil yield of the test oil palm plant further comprises applying a dominant model. Also in some examples, the predicting of palm oil yield of the test oil palm plant further comprises applying a recessive model.

The degree to which a particular SNP genotype of a SNP marker in candidate gene regions 1 to 5 can be useful for predicting palm oil yield of a test oil palm plant can depend on the source and breeding history of the breeding materials used to generate the population from which the test oil palm is sampled, including for example the extent to which one or more high-yield variant alleles that result in increases in palm oil yield have arisen within candidate gene regions 1 to 5 of the breeding materials and/or sources thereof used to generate the population, as well as the proximity of the one or more high-yield variant alleles to SNPs and the extent to which recombination has occurred between the SNPs and the high-yield variant alleles since the high-yield variant alleles arose. Factors such as proximity between a high-yield variant allele that promotes a high-oil-production trait and a SNP allele, a low number of generations since the high-yield variant allele arose, and a strong positive effect of the high-yield variant allele on palm oil production can tend to increase the degree to which of a particular SNP can be informative. These factors can vary, for example, depending on whether a high-yield variant allele is dominant or recessive, and thus whether a genotype model, a dominant model, or a recessive model may appropriately be applied with respect to a corresponding SNP allele. These factors also can vary, for example, between different populations generated by crosses of different individual palm plants.

The step of predicting palm oil yield of the test oil palm plant can be used advantageously not just to predict the palm oil yield of the test oil palm plant itself, but also to predict palm oil yields of progeny thereof. In this regard, oil palm breeders can use the method, as applied to a test oil palm plant that is a mother palm or a pollen donor, to determine possible SNP genotypes of progeny to be generated by crossing the test oil palm plant with another oil palm plant, and moreover can choose specific palms, i.e. the test oil palm plant and another specific oil palm plant that has been similarly characterized, to be crossed on this basis.

The method for predicting palm oil yield of a test oil palm plant can be used by focusing on particular candidate gene regions, or combinations thereof, with respect to test oil palm plants derived from particular breeding materials.

For example, in some examples the first candidate gene region is candidate gene region 1, and the population of oil palm plants comprises a Ulu Remis dura x AVROS pisifera population, a Johor Labis dura x AVROS pisifera population, a Ulu Remis dura x Dumpy AVROS pisifera population, or a combination thereof

Also, in some examples the first candidate gene region is candidate gene region 2, and the population of oil palm plants comprises a Ulu Remis dura x AVROS pisifera population, a Ulu Remis dura x Dumpy AVROS pisifera population, or a combination thereof.

Also, in some examples the first candidate gene region is candidate gene region 3, and the population of oil palm plants comprises a Dumpy AVROS population.

Also, in some examples the first candidate gene region is candidate gene region 4, and the population of oil palm plants comprises a Ulu Remis dura x AVROS pisifera population, a Johor Labis dura x AVROS pisifera population, or a combination thereof.

Also, in some examples the first candidate gene region is candidate gene region 5, and the population of oil palm plants comprises a Ulu Remis dura x AVROS pisifera population, a Ulu Remis dura x Dumpy AVROS pisifera population, or a combination thereof.

As noted above, crossing dura and pisifera gives rise to palms with a third fruit type, the tenera. As also noted, tenera are typically used as commercial planting materials. Accordingly, in some examples the test oil palm plant is a tenera candidate agricultural production plant. In some examples the population of oil palm plants comprises a Ulu Remis dura x AVROS pisifera population, and the test oil palm plant is a tenera candidate agricultural production plant. Also, in some examples the population of oil palm plants comprises a Johor Labis dura x AVROS pisifera population, and the test oil palm plant is a tenera candidate agricultural production plant. Also, in some examples the population of oil palm plants comprises a Ulu Remis dura x Dumpy AVROS pisifera population, and the test oil palm plant is a tenera candidate agricultural production plant.

As also noted above, oil palm breeding is primarily aimed at selecting for improved parental dura and pisifera breeding stock palms for production of superior tenera commercial planting materials. As also noted, parental dura breeding populations are generated by crossing among selected dura palms, whereas pisifera palms are normally female sterile and thus breeding populations thereof must be generated by crossing among selected teneras or by crossing selected teneras with selected pisiferas. Accordingly, in some examples the test oil palm plant is a plant for mother palm selection and propagation, a plant for introgressed mother palm selection and propagation, or a plant for pollen donor selection and propagation. In some examples, the population of oil palm plants comprises a Ulu Remis dura x Ulu Remis dura population, and the test oil palm plant is a plant for mother palm selection and propagation. Also in some examples, the population of oil palm plants comprises a Ulu Remis dura x Johor Labis dura population, and the test oil palm plant is a plant for introgressed mother palm selection and propagation. Also in some examples, the population of oil palm plants comprises a Johor Labis dura x Johor Labis dura population, and the test oil palm plant is a plant for mother palm selection and propagation. Also in some examples, the population of oil palm plants comprises an AVROS pisifera x AVROS tenera population, and the test oil palm plant is a plant for pollen donor selection and propagation. Also in some examples, the population of oil palm plants comprises an AVROS tenera x AVROS tenera population, and the test oil palm plant is a plant for pollen donor selection and propagation. Also in some examples, the population of oil palm plants comprises a Dumpy AVROS pisifera x Dumpy AVROS tenera population, and the test oil palm plant is a plant for pollen donor selection and propagation. Also in some examples, the population of oil palm plants comprises a Dumpy AVROS tenera x Dumpy AVROS tenera population, and the test oil palm plant is a plant for pollen donor selection and propagation.

The method for predicting palm oil yield of a test oil palm plant also can be carried out by determining additional SNP genotypes, comparing the additional SNP genotypes to corresponding reference genotypes indicative of the high-oil-production trait, and further predicting palm oil yield of the test oil palm plant based on the extent to which the additional SNP genotypes match the corresponding reference SNP genotypes. This is because each SNP genotype can reflect a high-yield variant allele that contributes to a high-oil-production trait additively and/or synergistically with respect to the others.

Accordingly, in some examples step (i) further comprises determining, from the sample of the test oil palm plant, at least a second SNP genotype of the test oil palm plant, the second SNP genotype corresponding to a second SNP marker, the second SNP marker (a) being located in a second candidate gene region for a high-oil-production trait and (b) being associated, after stratification and kinship correction, with the high-oil-production trait with a p-value <0.001 in the population or having a linkage disequilibrium r² value of at least 0.2 with respect to a second other SNP marker that is linked thereto and associated, after stratification and kinship correction, with the high-oil-production trait with a p-value <0.001 in the population. Moreover, in these examples step (ii) further comprises comparing the second SNP genotype of the test oil palm plant to a corresponding second reference SNP genotype indicative of the high-oil-production trait in the same genetic background as the population. In addition, in these examples the second candidate gene region corresponds to one of candidate gene regions 1 to 5, with the proviso that the first candidate gene region and the second candidate gene region correspond to different candidate gene regions. In some of these examples, step (iii) further comprises predicting palm oil yield of the test oil palm plant based on the extent to which the second SNP genotype of the test oil palm plant matches the corresponding second reference SNP genotype.

Also in some examples, step (i) further comprises determining, from the sample of the test oil palm plant, at least a third SNP genotype to a fifth SNP genotype of the test oil palm plant, the third SNP genotype to the fifth SNP genotype corresponding to a third SNP marker to a fifth SNP marker, respectively, the third SNP marker to the fifth SNP marker (a) being located in a third candidate gene region to a fifth candidate gene region, respectively, for the high-oil-production trait and (b) being associated, after stratification and kinship correction, with the high-oil-production trait with a p-value <0.001 in the population or having a linkage disequilibrium r² value of at least 0.2 with respect to a third other SNP marker to a fifth other SNP marker, respectively, that are linked thereto and associated, after stratification and kinship correction, with the high-oil-production trait with a p-value <0.001 in the population. Moreover, in these examples step (ii) further comprises comparing the third SNP genotype to the fifth SNP genotype of the test oil palm plant to a corresponding third reference SNP genotype to a corresponding fifth reference SNP genotype, respectively, indicative of the high-oil-production trait in the same genetic background as the population. In addition, in these examples the third candidate gene region to the fifth candidate gene region each correspond to one of candidate gene regions 1 to 5, with the proviso that the first candidate gene region to the fifth candidate gene region each correspond to different candidate gene regions. In some of these examples, step (iii) further comprises predicting palm oil yield of the test oil palm plant based on the extent to which the third SNP genotype to the fifth SNP genotype of the test oil palm plant match the corresponding third reference SNP genotype to the corresponding fifth reference SNP genotype, respectively.

Also provided is a method of selecting a high-palm-oil-yielding oil palm plant for agricultural production of palm oil. The method comprises a step of (a) predicting palm oil yield of a test oil palm plant. This step can be carried out according to the method described above, i.e. including a step of (i) determining, from a sample of a test oil palm plant of a population of oil palm plants, at least a first SNP genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first candidate gene region is a region of the oil palm genome corresponding to candidate gene regions 1 to 5, as described above. The method also comprises a step of (b) field planting the test oil palm plant for agricultural production of palm oil if the palm oil yield of the test oil palm plant is predicted to be higher than average for the population based on step (a).

Also provided is a method of selecting a high-palm-oil-yielding oil palm plant for cultivation in cell culture. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first SNP genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first candidate gene region is a region of the oil palm genome corresponding to candidate gene regions 1 to 5, as described above. The method also comprises a step of (b) subjecting at least one cell of the test oil palm plant to cultivation in cell culture if the palm oil yield of the test oil palm plant is predicted to be higher than average for the population based on step (a).

Also provided is a method of selecting a parental oil palm plant for use in breeding to obtain agricultural production plants or improved parental oil palm plants. As noted above, oil palm breeders can use the method, as applied to a test oil palm plant that is a mother palm or a pollen donor, to determine possible SNP genotypes of progeny to be generated by crossing the test oil palm plant with another oil palm plant, and moreover can choose specific palms, i.e. the test oil palm plant and another specific oil palm plant that has been similarly characterized, to be crossed on this basis. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first SNP genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first candidate gene region is a region of the oil palm genome corresponding to candidate gene regions 1 to 5, as described above. The method also comprises a step of (b) selecting the test oil palm plant for use in breeding if the palm oil yield of tenera progeny of the test oil palm plant is predicted to be higher than average for the population based on step (a).

As also noted above, in another example embodiment a method for predicting palm oil yield of a test oil palm plant is disclosed. The method comprises a step of (i) determining, from a sample of a test oil palm plant of a population of oil palm plants, at least a first single nucleotide polymorphism (also termed SNP) genotype of the test oil palm plant, as described above. Again, the SNP genotype of the test oil palm plant corresponds to the constitution of SNP alleles at a particular locus, or position, on each chromosome in which the locus occurs in the genome of the test oil palm plant, as described above.

The test oil palm plant can be an oil palm plant in any suitable form, as discussed above. Thus, for example, the test oil palm plant can be a seed, a seedling, a nursery phase plant, an immature phase plant, a cell culture plant, a zygotic embryo culture plant, or a somatic tissue culture plant. Also for example, the test oil palm plant can be a production phase plant, a mature palm, a mature mother palm, or a mature pollen donor.

Again, the population of oil palm plants from which the test oil palm plant is sampled can comprise any suitable population of oil palm plants. The population can be specified in terms of fruit type and/or identity of the breeding material from which the population was generated. Accordingly, in some examples the population of oil palm plants can comprise a Ulu Remis dura x AVROS pisifera population, a Johor Labis dura x AVROS pisifera population, a Ulu Remis dura x Dumpy AVROS pisifera population, a Dumpy AVROS population, or a combination thereof. Also in some examples the population of oil palm plants comprises a Ulu Remis dura x Ulu Remis dura population, a Ulu Remis dura x Johor Labis dura population, a Johor Labis dura x Johor Labis dura population, an AVROS pisifera x AVROS tenera population, an AVROS tenera x AVROS tenera population, a Dumpy AVROS pisifera x Dumpy AVROS tenera population, a Dumpy AVROS tenera x Dumpy AVROS tenera population, or a combination thereof.

Again, the sample of the test oil palm plant can comprise any organ, tissue, cell, or other part of the test oil palm plant that includes sufficient genomic DNA of the test oil palm plant to allow for determination of one or more SNP genotypes of the test oil palm plant, e.g. the first SNP genotype. Thus, for example, the sample can comprise a leaf tissue, among other organs, tissues, cells, or other parts. Also, determination of the at least first SNP genotype can be carried out by any suitable technique, as discussed above.

The first SNP genotype corresponds to a first SNP marker, as discussed above. Also, the first SNP marker is located in a first candidate gene region for a high-oil-production trait, as discussed above.

The high-oil-production trait relates to a trait of production of palm oil by the test oil palm plant upon reaching a mature state, e.g. reaching production phase, and upon being cultivated under conditions suitable for production of palm oil in a high amount, e.g. commercial cultivation, in an amount that is higher than average, with respect to the population of oil palm plants from which the test oil palm plant is sampled, also upon reaching a mature state and upon being cultivated under conditions suitable for production of palm oil in a high amount, as discussed above. Thus, for example, again considering a test oil palm plant that is a tenera oil palm plant, the high-oil-production trait can correspond, for example, to production of palm oil at greater than 3.67 tonnes of palm oil per hectare per year, i.e. above recent average yields for typical oil palm plants used in commercial production, which also are tenera oil palm plants, as discussed above. The high-oil production trait also can correspond, for example, to production of palm oil at greater than 10 tonnes of palm oil per hectare per year, i.e. above recent average yields for current best-progeny oil palm plants used in commercial production. The high-oil production trait also can correspond, for example, to production of palm oil at greater than 4, 5, 6, 7, 8, or 9 tonnes of palm oil per hectare per year, i.e. above yields that are intermediate between the recent average yields noted above. Also, considering a test oil palm plant that is a dura oil palm plant or a pisifera oil palm plant, the high-oil production trait can correspond to production of palm oil in correspondingly lower amounts, consistent with lower average yields obtained for dura and pisifera oil palm plants relative to tenera oil palm plants.

Again, the high-oil-production trait can comprise increased oil-to-dry mesocarp.

The first SNP marker is associated, after stratification and kinship correction, with the high-oil-production trait with a p-value <0.001 in the population or has a linkage disequilibrium r² value of at least 0.2 with respect to a first other SNP marker that is linked thereto and associated, after stratification and kinship correction, with the high-oil-production trait with a p-value <0.001 in the population, as discussed above.

Accordingly, for example, the first SNP marker being located in a first candidate gene region for a high-oil-production trait and being associated, after stratification and kinship correction, with the high-oil-production trait with a p-value <0.001 in the population can be a SNP marker for which association with the high-oil-production trait (i) has been confirmed based on a model that is not a naive model and/or (ii) would be confirmed based on a model that is not a naive model. Also for example, the first SNP marker being located in a first candidate gene region for a high-oil-production trait and being associated, after stratification and kinship correction, with the high-oil-production trait with a p-value <0.001 in the population can be a SNP marker for which association with the high-oil-production trait (i) has been confirmed based on a compressed mixed linear model with population parameters previously determined, carried out using principal component analysis and a group kinship matrix and/or (ii) would be confirmed based on a compressed mixed linear model with population parameters previously determined, carried out using principal component analysis and a group kinship matrix.

Accordingly, in some examples the first SNP marker is associated, after stratification and kinship correction, with the high-oil-production trait with a p-value <0.001 in the population. Also, in some examples the first SNP marker has a linkage disequilibrium r² value of at least 0.2 with respect to a first other SNP marker that is linked thereto and associated, after stratification and kinship correction, with the high-oil-production trait with a p-value <0.001 in the population. Also, in some examples both apply.

The first candidate gene region can be a region of the oil palm genome corresponding to:

(1) candidate gene region 6, comprising a gene encoding 1-Aminocyclopropane-1-Carboxylate Synthase 7 and extending from 4 kb upstream to 4 kb downstream of the gene encoding 1-Aminocyclopropane-1-Carboxylate Synthase 7;

(2) candidate gene region 7, comprising a gene encoding Mitochondrial Trans-2-Enoyl-CoA Reductase and extending from 4 kb upstream to 4 kb downstream of the gene encoding Mitochondrial Trans-2-Enoyl-CoA Reductase; or

(3) candidate gene region 8, comprising a gene encoding Chloroplastic Biotin Carboxyl Carrier Protein of Acetyl CoA Carboxylase and extending from 4 kb upstream to 4 kb downstream of the gene encoding Chloroplastic Biotin Carboxyl Carrier Protein of Acetyl CoA Carboxylase.

Considering candidate gene regions 6 to 8 in more detail, candidate gene region 6 includes a predicted transcription unit that includes 4 predicted exons, corresponding to the gene encoding 1-Aminocyclopropane-1-Carboxylate Synthase 7, and extends from 4 kb upstream of the transcription unit to 4 kb downstream of the transcription unit. 1-Aminocyclopropane-1-Carboxylate Synthase 7 catalyzes the rate-limiting step in biosynthesis of ethylene, which is responsible for fruit ripening, and is encoded by members of a divergent multigene family, the genes of which are differentially regulated by various environmental and developmental factors during plant growth. Genome-wide association studies revealed that candidate gene region 6 includes 10 SNP markers, of which one SNP marker, designated S_00000302_00166120 and having a SNP genotype major allele AG and a SNP genotype minor allele GG, is associated, after stratification and kinship correction, with the high-oil-production trait with a p-value <0.001 in the population, with respect to the Dumpy AVROS cluster. The S_00000302_00166120 SNP marker is located in candidate gene region 6 within the 5′ untranslated region of the gene encoding 1-Aminocyclopropane-1-Carboxylate Synthase 7, specifically 728 base pairs from the ATG start codon thereof, and has a SNP change corresponding to “T>C.”

Candidate gene region 7 includes a predicted transcription unit that includes 11 predicted exons, corresponding to the gene encoding Mitochondrial Trans-2-Enoyl-CoA Reductase, and extends from 4 kb upstream of the transcription unit to 4 kb downstream of the transcription unit. Trans-2-Enoyl-CoA Reductase of Euglena gracilis has been reported to be useful for increasing lipid content in plants based on overexpression. Genome-wide association studies revealed that candidate gene region 7 includes 25 SNP markers, of which one SNP marker, designated S_00002174_00010170 and having a SNP genotype major allele AG and a SNP genotype minor allele AA, is associated, after stratification and kinship correction, with the high-oil-production trait with a p-value <0.001 in the population, with respect to the Dumpy AVROS cluster and the UR_ALL cluster. The S_00002174_00010170 SNP marker is located in candidate gene region 7 within an intron of the gene encoding Mitochondrial Trans-2-Enoyl-CoA Reductase, specifically 128 base pairs from exon 3 and 148 base pairs from exon 4 thereof, and has a SNP change corresponding to “G/A.”

Candidate gene region 8 includes a predicted transcription unit that includes 9 predicted exons, corresponding to the gene encoding Chloroplastic Biotin Carboxyl Carrier Protein of Acetyl CoA Carboxylase, and extends from 4 kb upstream of the transcription unit to 4 kb downstream of the transcription unit. Chloroplastic Biotin Carboxyl Carrier Protein of Acetyl CoA Carboxylase is a component of acetyl-CoA carboxylase and plays a role in carboxylation thereby. Genome-wide association studies revealed that candidate gene region 8 includes 15 SNP markers, of which two SNP markers, the first designated

S_00018257_00003287 and having a SNP genotype major allele AA and a SNP genotype minor allele CC, and the second designated S_00018257_00006313 and having a SNP genotype major allele AA and a SNP genotype minor allele GG, are associated, after stratification and kinship correction, with the high-oil-production trait with a p-value <0.001 in the population, with respect to the AVROS cluster and the UR x AVROS cluster. The S_00018257_00003287 SNP marker is located in candidate gene region 8 within an intron of the gene encoding Chloroplastic Biotin Carboxyl Carrier Protein of Acetyl CoA Carboxylase, specifically 626 base pairs from exon 1, between exons 1 and 2, and has a SNP change corresponding to “T/G.” The S_00018257_00006313 SNP marker is located in candidate gene region 8 within an intron of the gene encoding Chloroplastic Biotin Carboxyl Carrier Protein of Acetyl CoA Carboxylase, specifically 364 base pairs from exon 3, between exons 2 and 3, and has a SNP change corresponding to “T/C.”

The method also comprises a step of (ii) comparing the first SNP genotype of the test oil palm plant to a corresponding first reference SNP genotype indicative of the high-oil-production trait in the same genetic background as the population, as discussed above.

The genetic background that is the same as the population can correspond, for example, to a population based on crossing oil palm plants of the same types as used to generate the population from which the test oil palm plant is sampled, e.g. a Ulu Remis dura x AVROS pisifera population, a Johor Labis dura x AVROS pisifera population, a Ulu Remis dura x Dumpy AVROS pisifera population, a Dumpy AVROS population, or a combination thereof, or a Ulu Remis dura x Ulu Remis dura population, a Ulu Remis dura x Johor Labis dura population, a Johor Labis dura x Johor Labis dura population, an AVROS pisifera x AVROS tenera population, an AVROS tenera x AVROS tenera population, a Dumpy AVROS pisifera x Dumpy AVROS tenera population, a Dumpy AVROS tenera x Dumpy AVROS tenera population, or a combination thereof. Again, the genetic background that is the same as the population also can correspond, for example, to a population based on crossing the same individual oil palm plants used to generate the population from which the test oil palm plant is sampled. The genetic background that is the same as the population also can correspond, for example, to the same actual population from which the test oil palm plant is sampled.

The first reference SNP genotype indicative of the high-oil-production trait in the same genetic background as the population can correspond to the same SNP as the first SNP genotype, i.e. both can correspond to the same polymorphic variation with respect to a single nucleotide that occurs at a particular locus of a particular chromosome, as discussed above. Also, the first reference SNP genotype can comprise one or more SNP alleles that, alone or together, indicate a higher likelihood that the test oil palm plant thereof exhibits, if mature, or will exhibit, upon reaching maturity, the high-oil-production trait, in comparison to oil palm plants of the same population that lack the one or more SNP alleles, as discussed above.

The method also comprises a step of (iii) predicting palm oil yield of the test oil palm plant based on the extent to which the first SNP genotype of the test oil palm plant matches the corresponding first reference SNP genotype, as discussed above. Thus, for example, the first SNP genotype of the test oil palm plant can match the corresponding first reference SNP genotype based on both SNP genotypes sharing at least a first SNP allele indicative of the high-oil-production trait in the same genetic background as the population. Again, in some examples the first SNP genotype and the first reference SNP genotype are heterozygous for the first allele indicative of the high-oil production trait, i.e. both have only one copy of the SNP allele. Also, in some examples the first SNP genotype and the first reference SNP genotype are homozygous for the first allele indicative of the high-oil production trait, i.e. both have two copies of the SNP allele. Also, in some examples the first SNP genotype is heterozygous for the first allele indicative of the high-oil production trait and the first reference SNP genotype is homozygous for the first allele indicative of the high-oil production trait. Also, in some examples the first SNP genotype is homozygous for the first allele indicative of the high-oil production trait and the first reference SNP genotype is heterozygous for the first allele indicative of the high-oil production trait.

Again, the step of predicting palm oil yield of the test oil palm plant can further comprise applying a model, such as a genotype model, a dominant model, or a recessive model, among others, in order to facilitate the predicting. Accordingly, in some examples, the predicting of palm oil yield of the test oil palm plant further comprises applying a genotype model. Also in some examples, the predicting of palm oil yield of the test oil palm plant further comprises applying a dominant model. Also in some examples, the predicting of palm oil yield of the test oil palm plant further comprises applying a recessive model.

Again, the step of predicting palm oil yield of the test oil palm plant can be used advantageously not just to predict the palm oil yield of the test oil palm plant itself, but also to predict palm oil yields of progeny thereof. Also, the method for predicting palm oil yield of a test oil palm plant can be used by focusing on particular candidate gene regions, or combinations thereof, with respect to test oil palm plants derived from particular breeding materials.

For example, in some examples the first candidate gene region is candidate gene region 6, and the population of oil palm plants comprises a Dumpy AVROS population.

Also, in some examples the first candidate gene region is candidate gene region 7, and the population of oil palm plants comprises a Dumpy AVROS population.

Also, in some examples the first candidate gene region is candidate gene region 8, and the population of oil palm plants comprises a Ulu Remis dura x AVROS pisifera population, a Johor Labis dura x AVROS pisifera population, or a combination thereof.

In some examples the test oil palm plant is a tenera candidate agricultural production plant, as discussed above. In some examples the population of oil palm plants comprises a Ulu Remis dura x AVROS pisifera population, and the test oil palm plant is a tenera candidate agricultural production plant. Also, in some examples the population of oil palm plants comprises a Johor Labis dura x AVROS pisifera population, and the test oil palm plant is a tenera candidate agricultural production plant. Also, in some examples the population of oil palm plants comprises a Ulu Remis dura x Dumpy AVROS pisifera population, and the test oil palm plant is a tenera candidate agricultural production plant.

In some examples the test oil palm plant is a plant for mother palm selection and propagation, a plant for introgressed mother palm selection and propagation, or a plant for pollen donor selection and propagation, as discussed above. In some examples, the population of oil palm plants comprises a Ulu Remis dura x Ulu Remis dura population, and the test oil palm plant is a plant for mother palm selection and propagation. Also in some examples, the population of oil palm plants comprises a Ulu Remis dura x Johor Labis dura population, and the test oil palm plant is a plant for introgressed mother palm selection and propagation. Also in some examples, the population of oil palm plants comprises a Johor Labis dura x Johor Labis dura population, and the test oil palm plant is a plant for mother palm selection and propagation. Also in some examples, the population of oil palm plants comprises an AVROS pisifera x AVROS tenera population, and the test oil palm plant is a plant for pollen donor selection and propagation. Also in some examples, the population of oil palm plants comprises an AVROS tenera x AVROS tenera population, and the test oil palm plant is a plant for pollen donor selection and propagation. Also in some examples, the population of oil palm plants comprises a Dumpy AVROS pisifera x Dumpy AVROS tenera population, and the test oil palm plant is a plant for pollen donor selection and propagation. Also in some examples, the population of oil palm plants comprises a Dumpy AVROS tenera x Dumpy AVROS tenera population, and the test oil palm plant is a plant for pollen donor selection and propagation.

The method for predicting palm oil yield of a test oil palm plant also can be carried out by determining additional SNP genotypes, comparing the additional SNP genotypes to corresponding reference genotypes indicative of the high-oil-production trait, and further predicting palm oil yield of the test oil palm plant based on the extent to which the additional SNP genotypes match the corresponding reference SNP genotypes, as discussed above.

Accordingly, in some examples step (i) further comprises determining, from the sample of the test oil palm plant, at least a second SNP genotype of the test oil palm plant, the second SNP genotype corresponding to a second SNP marker, the second SNP marker (a) being located in a second candidate gene region for a high-oil-production trait and (b) being associated, after stratification and kinship correction, with the high-oil-production trait with a p-value <0.001 in the population or having a linkage disequilibrium r² value of at least 0.2 with respect to a second other SNP marker that is linked thereto and associated, after stratification and kinship correction, with the high-oil-production trait with a p-value <0.001 in the population. Moreover, in these examples step (ii) further comprises comparing the second SNP genotype of the test oil palm plant to a corresponding second reference SNP genotype indicative of the high-oil-production trait in the same genetic background as the population. In addition, in these examples the second candidate gene region corresponds to one of candidate gene regions 6 to 8, with the proviso that the first candidate gene region and the second candidate gene region correspond to different candidate gene regions. In some of these examples, step (iii) further comprises predicting palm oil yield of the test oil palm plant based on the extent to which the second SNP genotype of the test oil palm plant matches the corresponding second reference SNP genotype.

Also in some examples, step (i) further comprises determining, from the sample of the test oil palm plant, at least a third SNP genotype of the test oil palm plant, the third SNP genotype corresponding to a third SNP marker, the third SNP marker (a) being located in a third candidate gene region for a high-oil-production trait and (b) being associated, after stratification and kinship correction, with the high-oil-production trait with a p-value <0.001 in the population or having a linkage disequilibrium r2 value of at least 0.2 with respect to a third other SNP marker that is linked thereto and associated, after stratification and kinship correction, with the high-oil-production trait with a p-value <0.001 in the population. Moreover, in these examples step (ii) further comprises comparing the third SNP genotype of the test oil palm plant to a corresponding third reference SNP genotype indicative of the high-oil-production trait in the same genetic background as the population. In addition, in these examples the third candidate gene region corresponds to one of candidate gene regions 6 to 8, with the proviso that the first candidate gene region to the third candidate gene region each correspond to different candidate gene regions. In some of these examples, step (iii) further comprises predicting palm oil yield of the test oil palm plant based on the extent to which the third SNP genotype of the test oil palm plant matches the corresponding third reference SNP genotype.

Also provided is a method of selecting a high-palm-oil-yielding oil palm plant for agricultural production of palm oil, as discussed above. The method comprises a step of (a) predicting palm oil yield of a test oil palm plant. This step can be carried out according to the method described above, i.e. including a step of (i) determining, from a sample of a test oil palm plant of a population of oil palm plants, at least a first SNP genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first candidate gene region is a region of the oil palm genome corresponding to candidate gene regions 6 to 8, as described above. The method also comprises a step of (b) field planting the test oil palm plant for agricultural production of palm oil if the palm oil yield of the test oil palm plant is predicted to be higher than average for the population based on step (a).

Also provided is a method of selecting a high-palm-oil-yielding oil palm plant for cultivation in cell culture, as discussed above. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first SNP genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first candidate gene region is a region of the oil palm genome corresponding to candidate gene regions 6 to 8, as described above. The method also comprises a step of (b) subjecting at least one cell of the test oil palm plant to cultivation in cell culture if the palm oil yield of the test oil palm plant is predicted to be higher than average for the population based on step (a).

Also provided is a method of selecting a parental oil palm plant for use in breeding to obtain agricultural production plants or improved parental oil palm plants, as discussed above. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first SNP genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first candidate gene region is a region of the oil palm genome corresponding to candidate gene regions 6 to 8, as described above. The method also comprises a step of (b) selecting the test oil palm plant for use in breeding if the palm oil yield of tenera progeny of the test oil palm plant is predicted to be higher than average for the population based on step (a).

The following examples are for purposes of illustration and are not intended to limit the scope of the claims.

### EXAMPLES

### Experimental Scope and Data

A combination of hypothesis-free genome-wide association studies, for identification of SNP markers associated with a high-oil-production-trait, and hypothesis-driven candidate gene region studies, for identification of candidate gene regions within the oil palm genome, was carried out. Objectives included identifying SNP markers that can be used to screen oil palm plants and identify those that exhibit high values of oil-to-dry mesocarp, and identifying genes that are highly associated with the trait of oil-to-dry mesocarp.

Specifically, 3670 oil palm plants, representing seven partially overlapping clusters of oil palm plants were subjected to SNP analysis. The seven partially overlapping clusters were as follows: a UR x AVROS cluster (n=1218), a Johor Labis x AVROS cluster (n=625), a UR x Dumpy AVROS cluster (n=1033), a Dumpy AVROS cluster (n=678), an AVROS cluster (n=1592), a UR_ALL cluster (n=2251), and a Nigerian dura x AVROS pisifera cluster (n=586). The sample selection was based on a good representation of oil-to-dry mesocarp variants and pedigree recorded by corresponding breeders. Total genomic DNA was isolated from unopened spear leaves using the DNAeasy (R) Plant Mini Kit (Qiagen, Limburg, Netherlands).

Regarding genetic stratification and population analysis, neighbor-joining (also termed NJ) tree was used to infer the genetic stratification of the genome wide associate study mapping populations. A Hamming's pairwise distance matrix for all SNP sites was calculated to plot the NJ tree. The genome-wide linkage disequilibrium decay rates in the various clusters were important to anticipate the requirements for suitable mapping resolution of the SNP for GWAS. The rate is defined as the chromosomal distance at which the average pairwise correlation coefficient (r²) dropped to the half of its maximum value. In this study, pairwise r² for all SNPs in a 1-Kb window were calculated and averaged across the whole genome based on composite method in the R package SNPrelate, in accordance with Zheng et al., Bioinformatics 28:3326-3328 (2012).

A total of 170,000 SNPs were selected from the oil palm genome for genotype screening of the 3670 palms. From the 170,000 SNPs, only 92,000 SNPs were found to be polymorphic, and of these 50,000 SNPs were determined to be located in or near candidate gene regions, each including a gene of interest (also termed GOI). Genes of interest were identified based on data generated from oil-palm transcriptomic, proteomic, and metabolomics platforms (also termed “omics” platforms). Domain checks and homology analyses were conducted to ensure that the genes were correctly annotated.

The 50,000 SNPs located in or near candidate gene regions were screened for association with a high-oil-production trait, specifically high oil-to-dry mesocarp, based on a p-value <0.001 in the population. Regarding phenotypic data compilation, oil-to-dry mesocarp is a direct measurement of crude palm oil (CPO) extracted from dry mesocarp tissue using a solvent. To measure oil-to-dry mesocarp, approximately 30 g of fertile fruits were randomly sampled per bunch from a minimum of three bunches per palm 4 years after field planting of the palms), resulting in a reliable mean oil-to-dry mesocarp. The oil-to-dry mesocarp differences between oil palms of the various clusters were tested for significance.

Boxplots of oil-to-dry mesocarp versus SNP genotype were plotted to identify SNP alleles that contribute to high or low values of oil-to-dry mesocarp. In accordance with the analysis, 223 SNPs were found to be associated with oil-to-dry mesocarp in at least one of the seven clusters of oil palm as tested, based on a p-value <0.001.

The 223 SNPs were further analyzed to distinguish SNPs that fall within intergenic regions, i.e. in regions between genes of interest, SNPs that might fall within long range linkage disequilibrium with candidate gene regions, and SNPs located within candidate gene regions including genes of interest that have been predicted with confidence. Of the 223 SNPs so identified, 116 SNPs fall within intergenic regions, 61 SNPs fall outside an isotig region, and only 13 SNPs are located within candidate gene regions including genes of interest that have been predicted with confidence, specifically within 4 kb upstream to 4 kb downstream of a given gene of interest.

The 13 SNPs so located represent eight candidate gene regions, designated candidate gene regions 1 to 8, each including a corresponding gene of interest, discussed further below. Accordingly, based on these findings, eight genes of interest were found to be associated with oil-to-dry mesocarp traits. Moreover, the 116 SNPs that fall within the intergenic regions and the 61 SNPs that fall outside an isotig region were checked for long-range linkage disequilibrium with the 13 SNPs.

### Candidate Gene Region 1

Candidate gene region 1 includes a gene encoding CBL-Interacting Protein Kinase 32. Specifically, candidate gene region 1 includes an 11 kb predicted transcription unit, designated SDt092051, that includes 18 predicted exons, and extends from 4 kb upstream of the transcription unit to 4 kb downstream of the transcription unit. Data from “omics” platforms, domain checks, and homology analyses indicated that the transcription unit corresponds to CBL-Interacting Protein Kinase 32. CBL-Interacting Protein Kinase 32 is a member of a calcium sensor protein family, the members of which interact with calcineurin B-like proteins, forming a complex and dynamic calcium-decoding signaling network, and are involved in abiotic stress regulation in maize, among other plants.

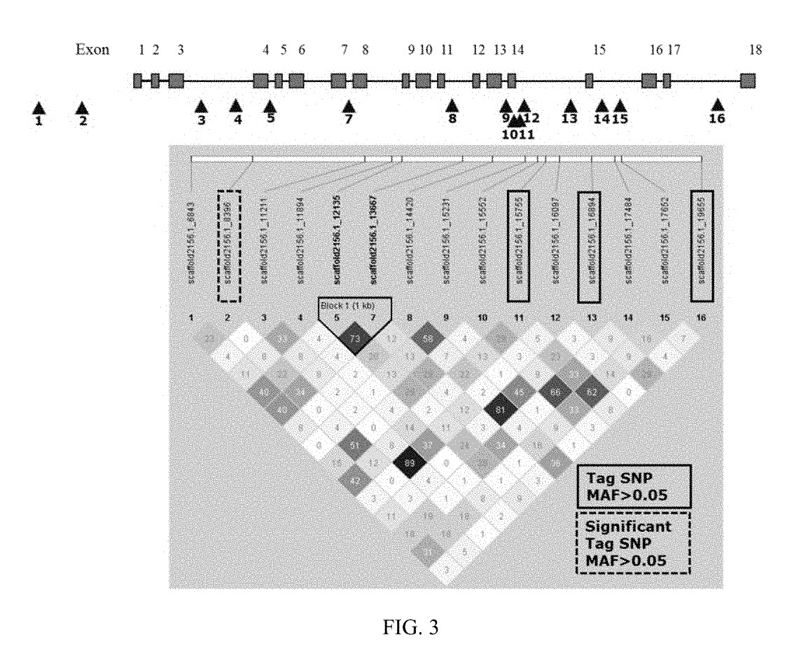

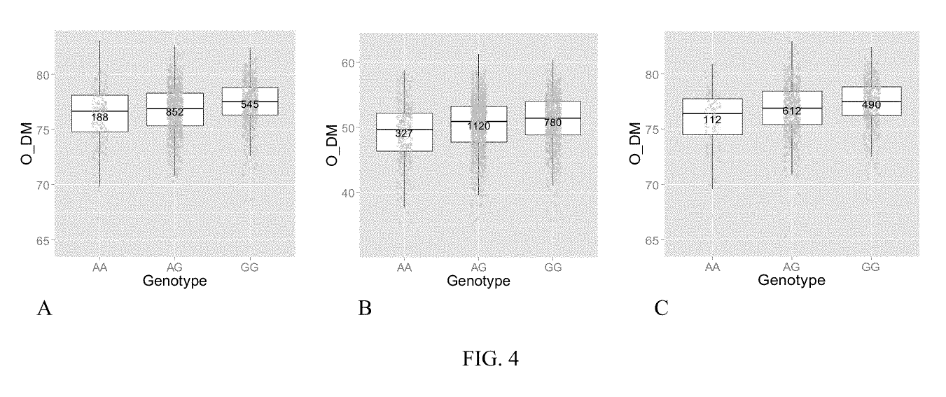

As shown in FIG. 3, genome-wide association studies revealed that candidate gene region 1 includes 15 SNP markers. As shown in FIG. 4, of the 15 SNP markers, one SNP marker, designated S_00002156_00008396 and having a SNP genotype major allele AG and a SNP genotype minor allele AA, is associated, after stratification and kinship correction, with the high-oil-production trait with a p-value <0.001 in the population, with respect to the AVROS cluster, the UR_ALL cluster, and the UR x AVROS cluster.

Considering the S_00002156_00008396 SNP marker in more detail, for the AVROS cluster, pairwise comparison of palms having the AA genotype (n=188), the AG genotype (n=852), and the GG genotype (n=545) by Student T-test indicated that palms having the GG genotype have a mean O/DM that is 0.6% higher than that of palms having the AG genotype (p-value=3.76E-08, i.e. 3.76×10⁻⁸), palms having the GG genotype have a mean O/DM that is 0.85% higher than that of palms having the AA genotype (p-value=1.04E-07), and palms having the AG genotype have a mean O/DM that is 0.5% higher than that of palms having the AA genotype (p-value=0.02) (FIG. 4A). Similarly, for the UR_ALL cluster, pairwise comparison of palms having the AA genotype (n=327), the AG genotype (n=1120), and the GG genotype (n=780) by Student T-test indicated that palms having the GG genotype have a mean O/DM that is 0.4% higher than that of palms having the AG genotype (p-value=7.34E-04), palms having the GG genotype have a mean O/DM that is 0.8% higher than that of palms having the AA genotype (p-value=5.09E-07), and palms having the AG genotype have a mean O/DM that is 0.4% higher than that of palms having the AA genotype (p-value=3.0E-03) (FIG. 4B). Also, for the UR x AVROS cluster, pairwise comparison of palms having the AA genotype (n=112), the AG genotype (n=612), and the GG genotype (n=490) by Student T-test indicated that palms having the GG genotype have a mean O/DM that is 0.6% higher than that of palms having the AG genotype (p-value=4.47E-05), palms having the GG genotype have a mean O/DM that is 1.1% higher than that of palms having the AA genotype (p-value=6.88E-07), and palms having the AG genotype have a mean O/DM that is 0.5% higher than that of palms having the AA genotype (p-value=2.0E-03) (FIG. 4C).

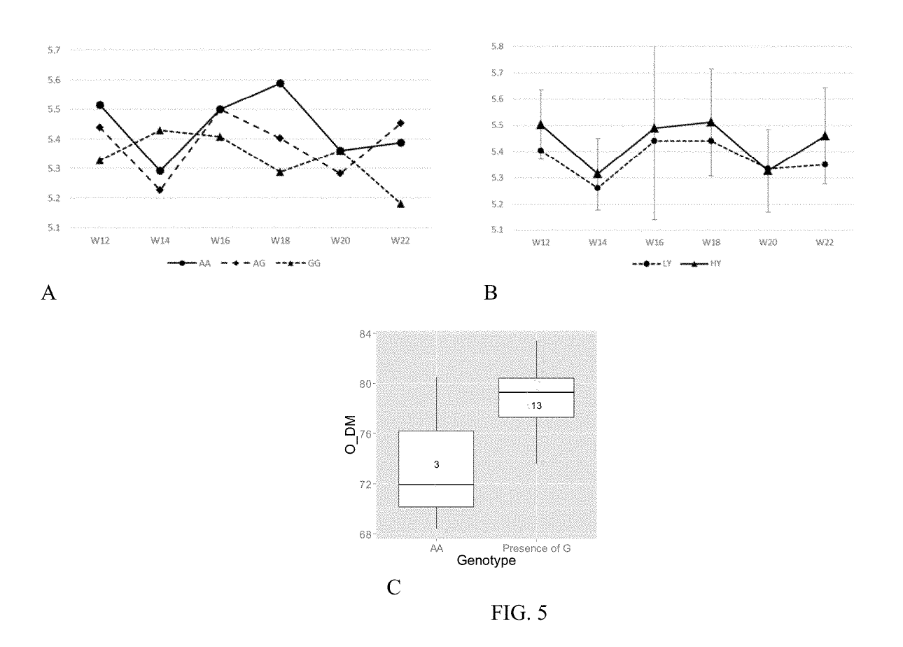

As shown in FIG. 5, comparative analysis of expression of the SDt092051 transcript, i.e. transcript of CBL-Interacting Protein Kinase 32, and mean O/DM for each of genotypes AA, AG, and GG, for eight high-yielding palms of these clusters and eight low-yielding palms of these clusters also indicated that palms having the GG or AG genotypes (n=13) exhibit a higher O/DM in comparison to palms having the AA genotype (n=3), specifically 7.4% higher mean O/DM (p-value=0.32).

Similar results also were obtained for the S_00002156_00008396 SNP marker with respect to oil-to-wet mesocarp (also termed O/WM or O_WM). For example, for the AVROS cluster, pairwise comparison of palms having the AA genotype (n=188), the AG genotype (n=852), and the GG genotype (n=545) by Student T-test indicated that palms having the GG genotype have a mean O/WM that is 0.9% higher than that of palms having the AG genotype (p-value=1.94E-05), palms having the GG genotype have a mean O/WM that is 2.3% higher than that of palms having the AA genotype (p-value=7.94E-10), and palms having the AG genotype have a mean O/WM that is 1.4% higher than that of palms having the AA genotype (p-value=9.63E-05). Similarly, for the UR_ALL cluster, the GG genotype yielded higher mean O/WM than the AG genotype or the AA genotype. Also, comparative analysis of mean O/WM for each of genotypes AA, AG, and GG, for eight high-yielding palms of these clusters and eight low-yielding palms of these clusters also indicated that palms having the GG genotype exhibit a higher O/WM in comparison to the palms having the AA and AG genotypes.

The S_00002156_00008396 SNP marker is located in candidate gene region 1 within the 5′ untranslated region of the gene encoding CBL-Interacting Protein Kinase 32, specifically 1861 base pairs from the ATG start codon thereof, and has a SNP change corresponding to “A>R (A/G).” Based on sequence analyses, it is predicted that a transcription factor binding site is provided overlapping the position of the S_00002156_00008396 SNP marker when the nucleotide at the SNP marker is A, and that no such transcription factor binding site is provided when the nucleotide at the SNP marker is G.

Taken together, the data for candidate gene region 1 and the S_00002156_00008396 SNP marker therein show consistency, coherency, and biological response. For example, the S_00002156_00008396 SNP marker yielded consistent results in more than one cluster of palms, namely in three clusters, and the GG genotype in particular was found to have the highest mean O/DM among the genotypes in all three of the clusters. Moreover, consistent results were observed regarding transcriptomics data for genotypes of the eight highest yielding palms and the eight lowest yielding palms, such that palms having the GG genotype have the highest mean O/DM. In addition, similar results were obtained with respect to O/WM, indicating coherency between the data for O/DM and O/WM and consistency in more than one cluster.

### Candidate Gene Region 2

Candidate gene region 2 includes a gene encoding Shaggy-Related Protein Kinase. Specifically, candidate gene region 2 includes a predicted transcription unit, designated SDt093033, that includes 11 predicted exons, and extends from 4 kb upstream of the transcription unit to 4 kb downstream of the transcription unit. Data from “omics” platforms, domain checks, and homology analyses indicated that the transcription unit corresponds to Shaggy-Related Protein Kinase. Shaggy-Related Protein Kinase is a member of Glycogen Synthase Kinase 3/SHAGGY-Like Kinase family, the members of which are involved in developmental processes of embryo, flower, and stomata, as well as wound response, and are involved in brassinosteroid signaling. Some members are upregulated transcriptionally in response to salt stress.

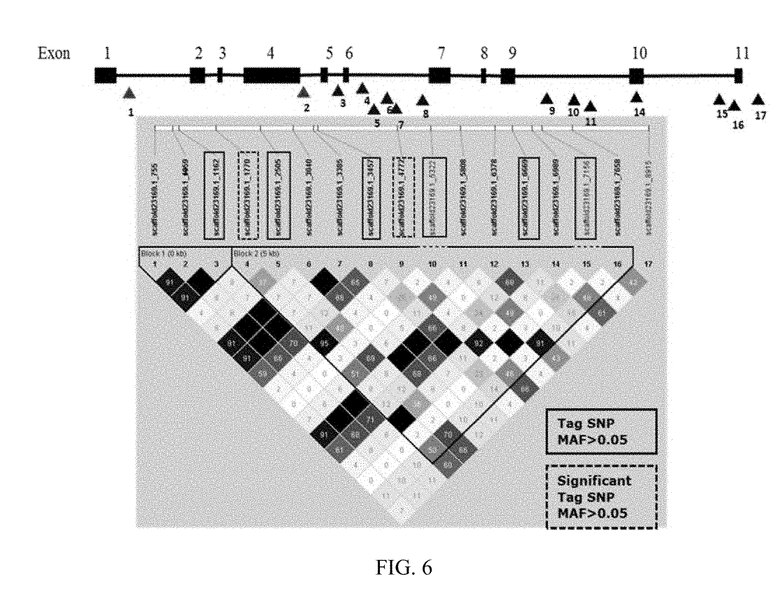

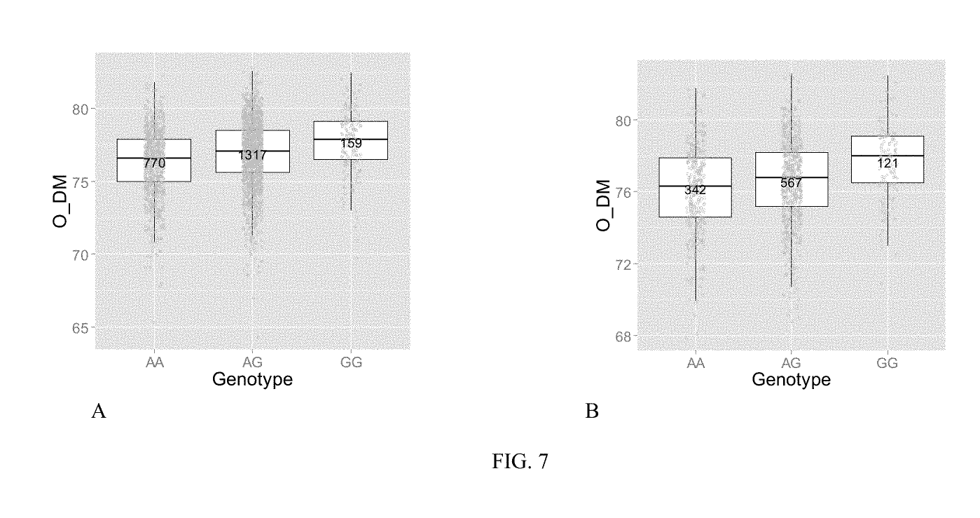

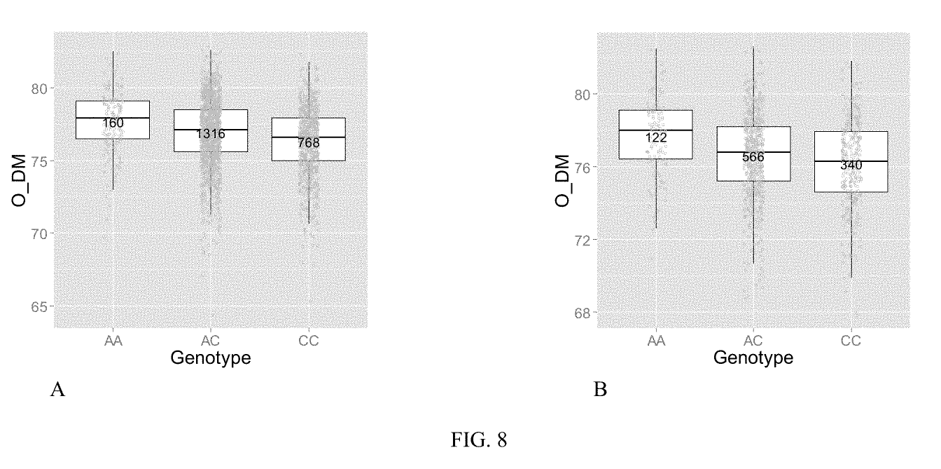

As shown in FIG. 6, genome-wide association studies revealed that candidate gene region 2 includes 17 SNP markers. As shown in FIG. 7 and FIG. 8, of the 17 SNP markers, two SNP markers, a first SNP marker designated S_000023169_00001770 and having a SNP genotype major allele AG and a SNP genotype minor allele GG, and a second SNP marker designated S_000023169_00004772 and having a SNP genotype major allele AC and a SNP genotype minor allele AA, are associated, after stratification and kinship correction, with the high-oil-production trait with a p-value <0.001 in the population, with respect to the UR_ALL cluster and the UR x Dumpy AVROS cluster.

Considering the S_000023169_00001770 SNP marker in more detail, for the UR_ALL cluster, pairwise comparison of palms having the AA genotype (n=770), the AG genotype (n=1317), and the GG genotype (n=159) by Student T-test indicated that palms having the GG genotype have a mean O/DM that is 0.8% higher than that of palms having the AG genotype (p-value=3.44E-05), palms having the GG genotype have a mean O/DM that is 1.3% higher than that of palms having the AA genotype (p-value=7.88E-12), and palms having the AG genotype have a mean O/DM that is 0.5% higher than that of palms having the AA genotype (p-value=1.98E-08) (FIG. 7A). Similarly, for the UR x Dumpy AVROS cluster, pairwise comparison of palms having the AA genotype (n=342), the AG genotype (n=567), and the GG genotype (n=121) by Student T-test indicated that palms having the GG genotype have a mean O/DM that is 1.2% higher than that of palms having the AG genotype (p-value=1.93E-06), palms having the GG genotype have a mean O/DM that is 1.7% higher than that of palms having the AA genotype (p-value=5.95E-10), and palms having the AG genotype have a mean O/DM that is 0.5% higher than that of palms having the AA genotype (p-value=7.33E-03) (FIG. 7B).

Considering the S_000023169_00004772 SNP marker in more detail, for the UR_ALL cluster, pairwise comparison of palms having the AA genotype (n=160), the AC genotype (n=1316), and the CC genotype (n=768) by Student T-test indicated that palms having the AA genotype have a mean O/DM that is 0.8% higher than that of palms having the AC genotype (p-value=3.99E-05), palms having the AA genotype have a mean O/DM that is 1.3% higher than that of palms having the CC genotype (p-value=8.82E-12), and palms having the AC genotype have a mean O/DM that is 0.5% higher than that of palms having the CC genotype (p-value=1.99E-08) (FIG. 8A). Similarly, for the UR x Dumpy AVROS cluster, pairwise comparison of palms having the AA genotype (n=122), the AC genotype (n=566), and the CC genotype (n=340) by Student T-test indicated that palms having the AA genotype have a mean O/DM that is 1.2% higher than that of palms having the AC genotype (p-value=2.25E-06), palms having the AA genotype have a mean O/DM that is 1.7% higher than that of palms having the CC genotype (p-value=8.82E-12), and palms having the AC genotype have a mean O/DM that is 0.5% higher than that of palms having the CC genotype (p-value=7.24E-03) (FIG. 8B).

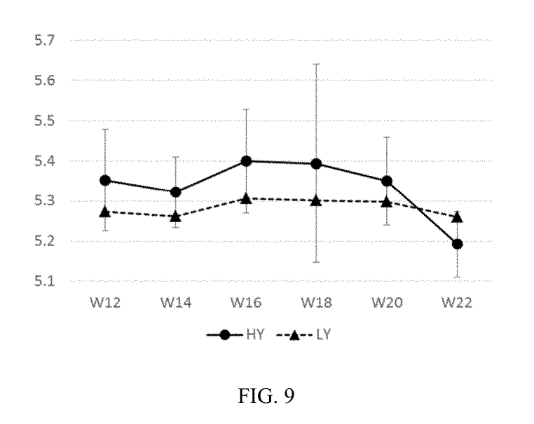

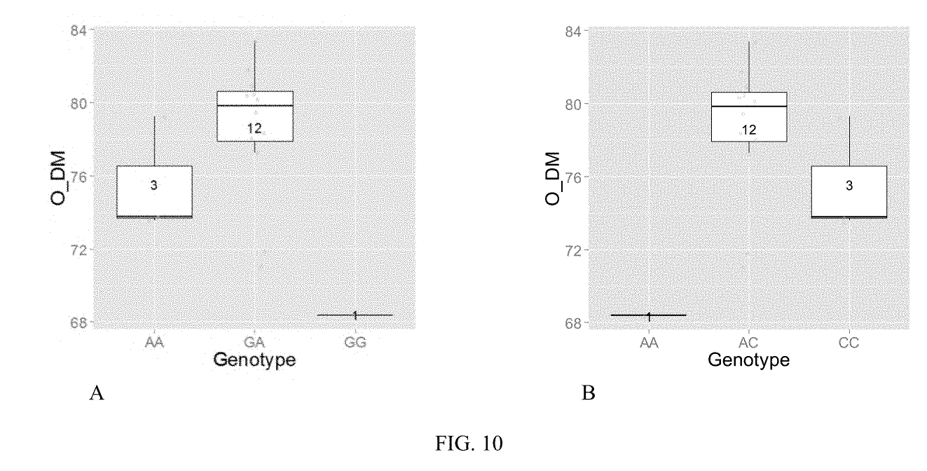

As shown in FIG. 9 and FIG. 10, comparative analysis of expression of the SDt093033 transcript, i.e. transcript of Shaggy-Related Protein Kinase, and mean O/DM for each of genotypes AA, AG, and GG of the S_000023169_00001770 SNP marker and for each of genotypes AA, AC, and CC of the S_000023169_00004772 SNP marker, for eight high-yielding palms of these clusters and eight low-yielding palms of these clusters also indicated that the SDt093033 transcript is upregulated in the highest yielding palms at weeks 12 to 16 after anthesis (FIG. 9), and that a similar pattern regarding O/DM was observed as for the association data regarding the S_000023169_00001770 SNP marker (FIG. 10A) and the S_000023169_00004772 SNP marker (FIG. 10B), although the sample sizes were small.

Similar results also were obtained for the S_000023169_00001770 SNP marker with respect to mesocarp per fruit (also termed M/F or M_F), shell per fruit (also termed S/F or S_F), kernel per fruit (also termed K/F or K_F), such that for the UR_ALL cluster and the UR x Dumpy AVROS cluster the GG genotype yielded lower M/F, higher S/F, and lower K/F than the AG genotype or the AA genotype. Similar results also were obtained for the S_000023169_00004772 SNP marker with respect to mesocarp per fruit, shell per fruit, and kernel per fruit, such that for the UR_ALL cluster and the UR x Dumpy AVROS cluster the AA genotype yielded lower M/F, higher S/F, and lower K/F than the AC genotype or the CC genotype.

The S_000023169_00001770 SNP marker is located in candidate gene region 2 within an intron of the gene encoding Shaggy-Related Protein Kinase, specifically 240 base pairs from exon 7, between exons 6 and 7, and has a SNP change corresponding to “A/G.” Sequence analysis indicates predicted splicing. The S_000023169_00004772 SNP marker is located in candidate gene region 2 within an intron of the gene encoding Shaggy-Related Protein Kinase, specifically 556 base pairs from exon 10, between exons 9 and 10, and has a SNP change corresponding to “A/C.” Sequence analysis indicates no predicted splicing.

Taken together, the data for candidate gene region 2 and the S_000023169_00001770 SNP marker and the S_000023169_00004772 SNP marker therein show consistency, coherency, and biological response. For example, the S_000023169_00001770 SNP marker and the S_000023169_00004772 SNP marker yielded consistent results in more than one cluster of palms, namely in two clusters. For the S_000023169_00001770 SNP marker, the GG genotype in particular was found to have the highest mean O/DM among the genotypes in the two clusters, and for the S_000023169_00004772 SNP marker, the AA genotype in particular was found to have the highest mean O/DM among the genotypes in the two clusters. Moreover, consistent results were observed regarding transcriptomics data for genotypes of the eight highest yielding palms and the eight lowest yielding palms. In addition, similar results were obtained with respect to mesocarp per fruit, shell per fruit, and kernel per fruit, indicating coherency between the data for O/DM, M/F, S/F, and K/F and consistency in more than one cluster.

### Candidate Gene Region 3

Candidate gene region 3 includes a gene encoding Probable Receptor-Like Protein Kinase. Specifically, candidate gene region 3 includes a predicted transcription unit, designated SDt026153, that includes 5 predicted exons, and extends from 4 kb upstream of the transcription unit to 4 kb downstream of the transcription unit. Data from “omics” platforms, domain checks, and homology analyses indicated that the transcription unit corresponds to Probable Receptor-Like Protein Kinase. Probable Receptor-Like Protein Kinase is a member of Receptor-Like Protein Kinase family, the members of which are involved in signal transduction of abscisic acid in Arabidopsis and response toward environmental stress.

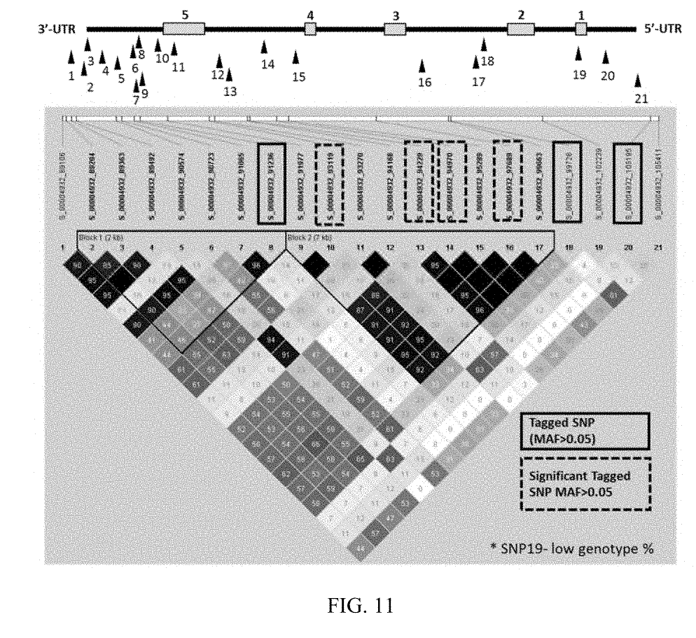

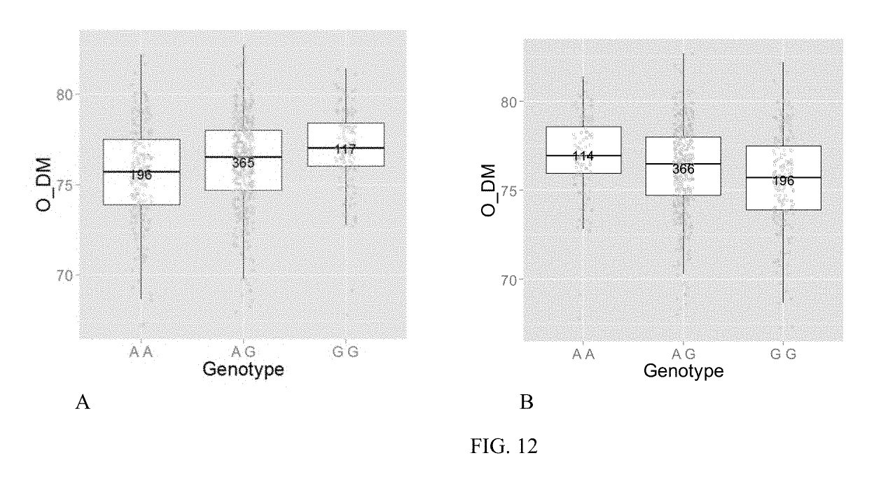

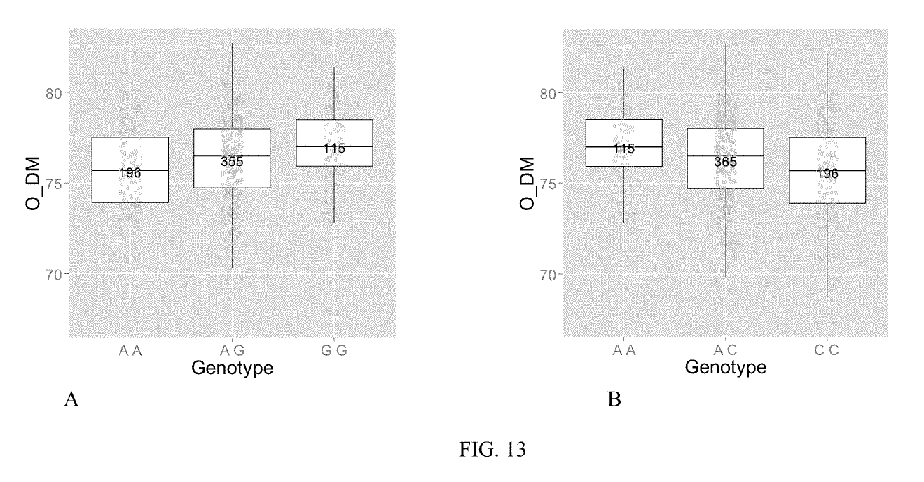

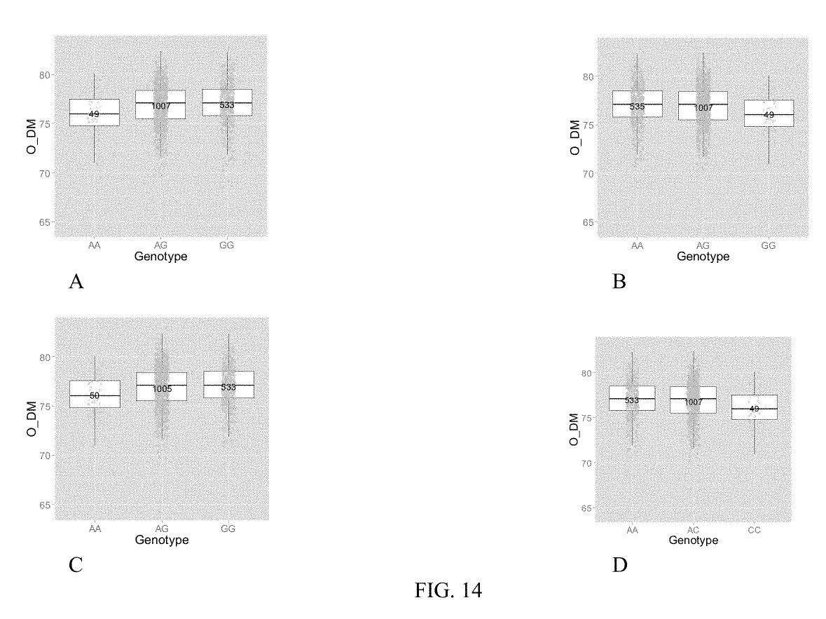

As shown in FIG. 11, genome-wide association studies revealed that candidate gene region 3 includes 21 SNP markers. As shown in FIG. 12, FIG. 13, and FIG. 14, of the 21 SNP markers, four SNP markers, a first SNP marker designated S_000004932_00093119 and having a SNP genotype major allele AG and a SNP genotype minor allele GG, a second SNP marker designated S_000004932_00094229 and having a SNP genotype major allele AG and a SNP genotype minor allele AA, a third SNP marker designated S_000004932_00094970 and having a SNP genotype major allele AG and a SNP genotype minor allele GG, and a fourth SNP marker designated S_000004932_00097689 and having a SNP genotype major allele AC and a SNP genotype minor allele AA are associated, after stratification and kinship correction, with the high-oil-production trait with a p-value <0.001 in the population, with respect to the Dumpy AVROS cluster. Moreover, as also shown, similar results were obtained with respect to the AVROS cluster, albeit not with a p-value <0.001.

Considering the S_000004932_00093119 SNP marker in more detail, for the Dumpy AVROS cluster, pairwise comparison of palms having the AA genotype (n=196), the AG genotype (n=365), and the GG genotype (n=117) by Student T-test indicated that palms having the GG genotype have a mean O/DM that is 0.5% higher than that of palms having the AG genotype (p-value=2.30E-03), palms having the GG genotype have a mean O/DM that is 1.3% higher than that of palms having the AA genotype (p-value=1.83E-06), and palms having the AG genotype have a mean O/DM that is 0.8% higher than that of palms having the AA genotype (p-value=7.38E-03) (FIG. 12A). Similarly, for the AVROS cluster, pairwise comparison of palms having the AA genotype (n=49), the AG genotype (n=1007), and the GG genotype (n=533) by Student T-test indicated that palms having the GG genotype have a mean O/DM that is 0% higher than that of palms having the AG genotype (p-value=0.273), palms having the GG genotype have a mean O/DM that is 1.1% higher than that of palms having the AA genotype (p-value=6.15E-03), and palms having the AG genotype have a mean O/DM that is 1.1% higher than that of palms having the AA genotype (p-value=6.15E-03) (FIG. 14A).

Considering the S_000004932_00094229 SNP marker in more detail, for the Dumpy AVROS cluster, pairwise comparison of palms having the AA genotype (n=114), the AG genotype (n=366), and the GG genotype (n=196) by Student T-test indicated that palms having the AA genotype have a mean O/DM that is 0.45% higher than that of palms having the AG genotype (p-value=3.04E-03), palms having the AA genotype have a mean O/DM that is 1.25% higher than that of palms having the GG genotype (p-value=2.84E-06), and palms having the AG genotype have a mean O/DM that is 0.8% higher than that of palms having the GG genotype (p-value=7.27E-03) (FIG. 12B). Similarly, for the AVROS cluster, pairwise comparison of palms having the AA genotype (n=535), the AG genotype (n=1007), and the GG genotype (n=49) by Student T-test indicated that palms having the AA genotype have a mean O/DM that is 0% higher than that of palms having the AG genotype (p-value=0.257), palms having the AA genotype have a mean O/DM that is 1.1% higher than that of palms having the GG genotype (p-value=5.93E-03), and palms having the AG genotype have a mean O/DM that is 1.1% higher than that of palms having the GG genotype (p-value=1.64E-02) (FIG. 14B).

Considering the S_000004932_00094970 SNP marker in more detail, for the Dumpy AVROS cluster, pairwise comparison of palms having the AA genotype (n=196), the AG genotype (n=355), and the GG genotype (n=115) by Student T-test indicated that palms having the GG genotype have a mean O/DM that is 0.5% higher than that of palms having the AG genotype (p-value=3.58E-03), palms having the GG genotype have a mean O/DM that is 1.3% higher than that of palms having the AA genotype (p-value=2.47E-06), and palms having the AG genotype have a mean O/DM that is 0.8% higher than that of palms having the AA genotype (p-value=6.28E-03) (FIG. 13A). Similarly, for the AVROS cluster, pairwise comparison of palms having the AA genotype (n=50), the AG genotype (n=1005), and the GG genotype (n=533) by Student T-test indicated that palms having the GG genotype have a mean O/DM that is 0% higher than that of palms having the AG genotype (p-value=0.284), palms having the GG genotype have a mean O/DM that is 1.05% higher than that of palms having the AA genotype (p-value=1.13E-02), and palms having the AG genotype have a mean O/DM that is 1.05% higher than that of palms having the AA genotype (p-value=2.81E-02) (FIG. 14C).

Considering the S_000004932_00097689 SNP marker in more detail, for the Dumpy AVROS cluster, pairwise comparison of palms having the AA genotype (n=115), the AC genotype (n=365), and the CC genotype (n=196) by Student T-test indicated that palms having the AA genotype have a mean O/DM that is 0.5% higher than that of palms having the AC genotype (p-value=2.72E-03), palms having the AA genotype have a mean O/DM that is 1.3% higher than that of palms having the CC genotype (p-value=2.40E-06), and palms having the AC genotype have a mean O/DM that is 0.8% higher than that of palms having the CC genotype (p-value=7.38E-03) (FIG. 13B). Similarly, for the AVROS cluster, pairwise comparison of palms having the AA genotype (n=533), the AC genotype (n=1007), and the CC genotype (n=49) by Student T-test indicated that palms having the AA genotype have a mean O/DM that is 0% higher than that of palms having the AC genotype (p-value=0.273), palms having the AA genotype have a mean O/DM that is 1.1% higher than that of palms having the CC genotype (p-value=6.15E-03), and palms having the AC genotype have a mean O/DM that is 1.1% higher than that of palms having the CC genotype (p-value=1.64E-02) (FIG. 14D).

The S_000004932_00093119 SNP marker is located in candidate gene region 3 within the 3′-untranslated region of the gene encoding Probable Receptor-Like Protein Kinase, specifically 288 base pairs from exon 5, and has a SNP change corresponding to “A/G.” The S_000004932_00094229 SNP marker is located in candidate gene region 3 within an intron of the gene encoding Probable Receptor-Like Protein Kinase, specifically 277 base pairs from exon 5, between exons 4 and 5, and has a SNP change corresponding to “C/T.” The S_000004932_00094970 SNP marker is located in candidate gene region 3 within an intron of the gene encoding Probable Receptor-Like Protein Kinase, specifically 625 base pairs from exon 4, between exons 4 and 5, and has a SNP change corresponding to “T/C.” The S_000004932_00097689 SNP marker is located in candidate gene region 3 within an intron of the gene encoding Probable Receptor-Like Protein Kinase, specifically 673 base pairs from exon 3, between exons 2 and 3, and has a SNP change corresponding to “G/T.”

Taken together, the data for candidate gene region 3 and the S_000004932_00093119 SNP marker, the S_000004932_00094229 SNP marker, the S_000004932_00094970 SNP marker, and the S_000004932_00097689 SNP marker therein show consistency, coherency, and biological response. For example, each of the S_000004932_00093119 SNP marker, the S_000004932_00094229 SNP marker, the S_000004932_00094970 SNP marker, and the S_000004932_00097689 SNP marker yielded consistent results in more than one cluster of palms, namely in two clusters, albeit with p-values <0.001 for only one of the two clusters. For the S_000004932 00093119 SNP marker, the GG genotype in particular was found to have the highest mean O/DM among the genotypes in the two clusters, for the S_000004932_00094229 SNP marker, the AA genotype in particular was found to have the highest mean O/DM among the genotypes in the two clusters, for the S_000004932_00094970 SNP marker, the GG genotype in particular was found to have the highest mean O/DM among the genotypes in the two clusters, and for the S_000004932_00097689 SNP marker, the AA genotype in particular was found to have the highest mean O/DM among the genotypes in the two clusters.

### Candidate Gene Region 4

Candidate gene region 4 includes a gene encoding Tau Class Glutathione S-Transferase. Specifically, candidate gene region 4 includes a predicted transcription unit, designated SDt081517, that includes 2 predicted exons, and extends from 4 kb upstream of the transcription unit to 4 kb downstream of the transcription unit. Data from “omics” platforms, domain checks, and homology analyses indicated that the transcription unit corresponds to Tau Class Glutathione S-Transferase. Tau Class Glutathione S-Transferase is a member of the Glutathione S-Transferase family, the members of which catalyze conjugation of compounds to glutathione and target the compounds for storage in vacuoles or apoplasts. The compounds can include products of oxidative stress, carcinogens, and/or environmental toxins.

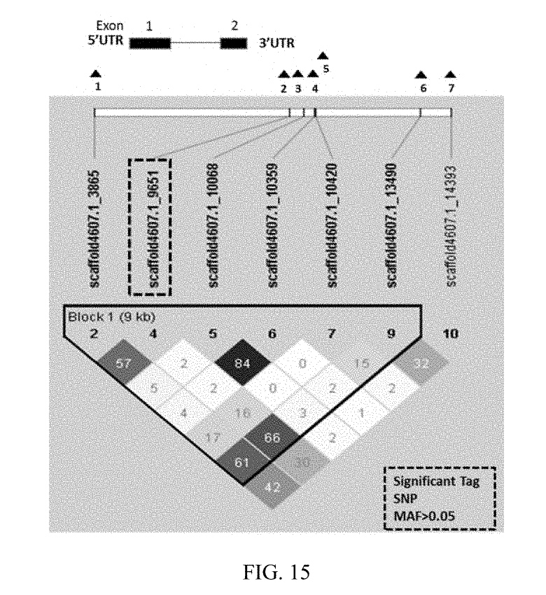

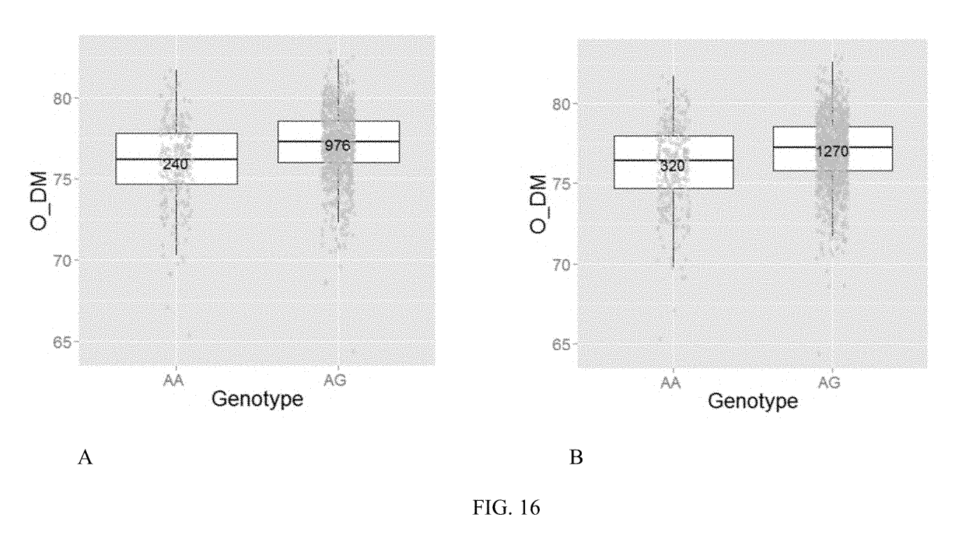

As shown in FIG. 15, genome-wide association studies revealed that candidate gene region 4 includes seven SNP markers. As shown in FIG. 16, of the seven SNP markers, one SNP marker, designated S_00004607_00009651 and having a SNP genotype major allele AG and a SNP genotype minor allele AA, is associated, after stratification and kinship correction, with the high-oil-production trait with a p-value <0.001 in the population, with respect to the UR x AVROS cluster and the AVROS cluster.

Considering the S_00004607_00009651 SNP marker in more detail, for UR x AVROS cluster, pairwise comparison of palms having the AA genotype (n=240) and the AG genotype (n=976) by Student T-test indicated that palms having the AG genotype have a mean O/DM that is 1.1% higher than that of palms having the AA genotype (p-value=6.14E-10) (FIG. 16A). Similarly, for the AVROS cluster, pairwise comparison of palms having the AA genotype (n=320) and the AG genotype (n=1270) by Student T-test indicated that palms having the AG genotype have a mean O/DM that is 0.8% higher than that of palms having the AA genotype (p-value=8.39E-09) (FIG. 16B).

Comparative analysis of expression of the SDt081517 transcript, i.e. transcript of Tau Class Glutathione S-Transferase, and mean O/DM for genotypes AA and AG, for eight high-yielding palms of these clusters and eight low-yielding palms of these clusters also indicated that palms having the AG genotypes (n=13) exhibit a higher O/DM in comparison to palms having the AA genotype (n=3), specifically 7.4% higher mean O/DM (p-value=0.34).

The S_00004607_00009651 SNP marker is located in candidate gene region 4 within the 3′ untranslated region of the gene encoding Tau Class Glutathione S-Transferase, specifically 3110 base pairs from the stop codon thereof, and has a SNP change corresponding to “A/G.”

Taken together, the data for candidate gene region 4 and the S_00004607_00009651 SNP marker therein show consistency, coherency, and biological response. For example, the S_00004607_00009651 SNP marker yielded consistent results in more than one cluster of palms, namely in two clusters, and the AG genotype in particular was found to have the highest mean O/DM among the genotypes in the two clusters.

### Candidate Gene Region 5

Candidate gene region 5 includes a gene encoding Cinnamoyl-CoA Reductase. Specifically, candidate gene region 5 includes a predicted transcription unit, designated SDt076624, that includes 6 predicted exons, and extends from 4 kb upstream of the transcription unit to 4 kb downstream of the transcription unit. Data from “omics” platforms, domain checks, and homology analyses indicated that the transcription unit corresponds to Cinnamoyl-CoA Reductase. Cinnamoyl-CoA Reductase catalyzes the first specific step in the synthesis of monomers of lignin, specifically reaction of cinnamaldehyde, CoA, and NADP+ to cinnamoyl-CoA, NADPH, and H+.

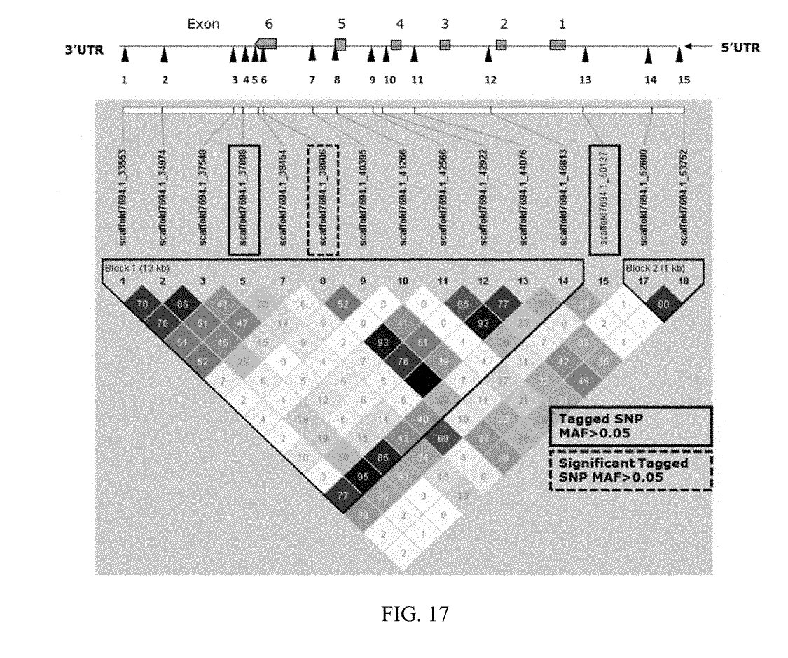

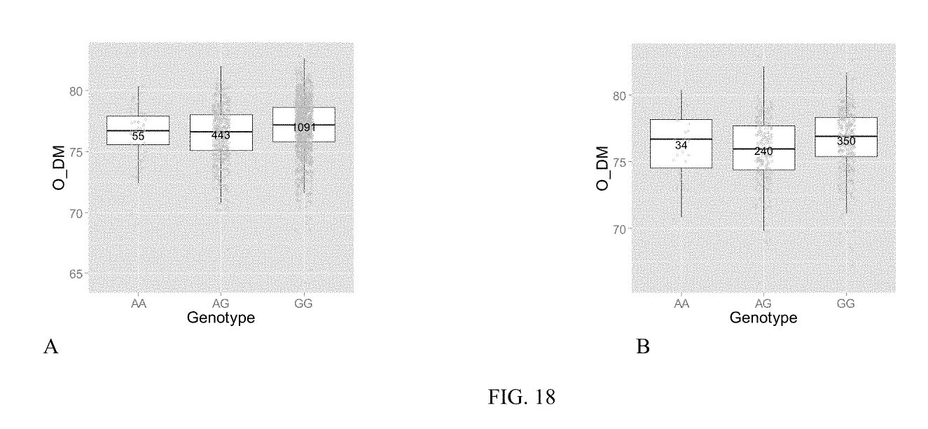

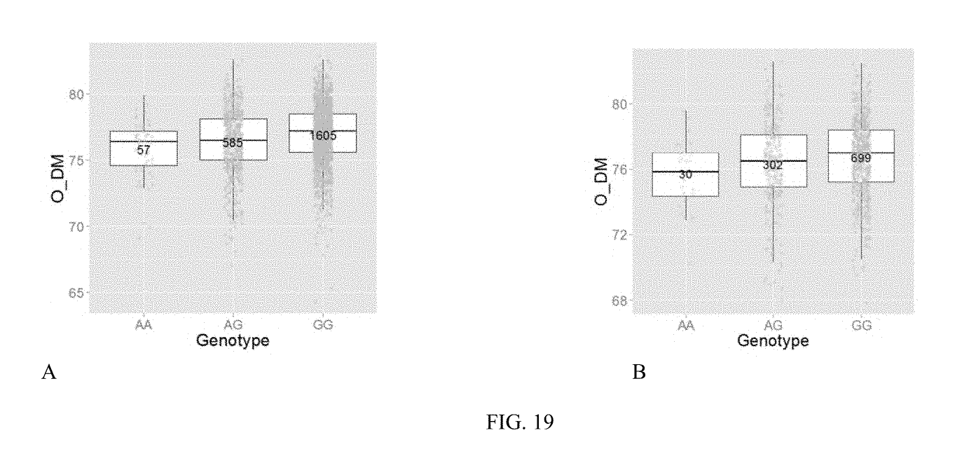

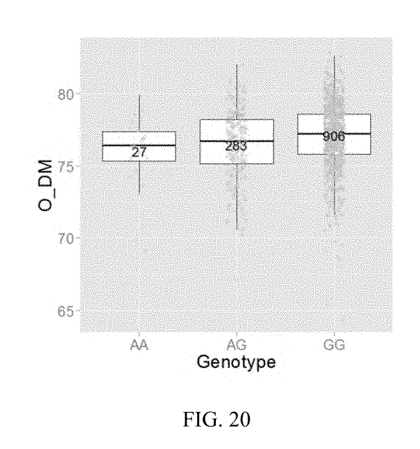

As shown in FIG. 17, genome-wide association studies revealed that candidate gene region 5 includes 15 SNP markers. As shown in FIG. 18, FIG. 19, and FIG. 20, of the 15 SNP markers, one SNP marker, designated S_00007694_00038606 and having a SNP genotype major allele GG and a SNP genotype minor allele AA, is associated, after stratification and kinship correction, with the high-oil-production trait with a p-value <0.001 in the population, with respect to the AVROS cluster, the Johor Labis x AVROS cluster, the UR_ALL cluster, and the UR x AVROS cluster. Moreover, as also shown, similar results were obtained with respect to the UR x Dumpy AVROS cluster, albeit not with a p-value <0.001.

Considering the S_00007694_00038606 SNP marker in more detail, for the AVROS cluster, pairwise comparison of palms having the AA genotype (n=55), the AG genotype (n=443), and the GG genotype (n=1091) by Student T-test indicated that palms having the GG genotype have a mean O/DM that is 0.84% higher than that of palms having the AG genotype (p-value=5.57E-07), and palms having the GG genotype have a mean O/DM that is 0.94% higher than that of palms having the AA genotype (p-value=0.02) (FIG. 18A). Similarly, for the Johor Labis x AVROS cluster, pairwise comparison of palms having the AA genotype (n=34), the AG genotype (n=240), and the GG genotype (n=350) by Student T-test indicated that palms having the GG genotype have a mean O/DM that is 0.95% higher than that of palms having the AG genotype (p-value=4.22E-04) (FIG. 18B). Also, for the UR_ALL cluster, pairwise comparison of palms having the AA genotype (n=57), the AG genotype (n=585), and the GG genotype (n=1605) by Student T-test indicated that palms having the GG genotype have a mean O/DM that is 0.73% higher than that of palms having the AG genotype (p-value=9.82E-07), and palms having the GG genotype have a mean O/DM that is 1.43% higher than that of palms having the AA genotype (p-value=4.44E-04) (FIG. 19A). Also, for the UR x Dumpy AVROS cluster, pairwise comparison of palms having the AA genotype (n=30), the AG genotype (n=302), and the GG genotype (n=699) by Student T-test indicated that palms having the GG genotype have a mean O/DM that is 0.54% higher than that of palms having the AG genotype (p-value=0.01), and palms having the GG genotype have a mean O/DM that is 1.34% higher than that of palms having the AA genotype (p-value=0.02) (FIG. 19B). Also, for the UR x AVROS cluster, pairwise comparison of palms having the AA genotype (n=27), the AG genotype (n=283), and the GG genotype (n=906) by Student T-test indicated that palms having the GG genotype have a mean O/DM that is 0.86% higher than that of palms having the AG genotype (p-value=2.61E-05), and palms having the GG genotype have a mean O/DM that is 1.31% higher than that of palms having the AA genotype (p-value=0.02) (FIG. 20).

Similar results also were obtained for the S_00007694_00038606 SNP marker with respect to mesocarp per fruit, shell per fruit, and kernel per fruit, such that for the UR x Dumpy AVROS cluster and the UR x AVROS cluster the GG genotype yielded lower M/F, higher S/F, and higher K/F than the AG genotype or the AA genotype.

Comparative analysis of expression of the SDt076624 transcript, i.e. transcript of Cinnamoyl-CoA Reductase, and mean O/DM for genotypes AG and GG, for eight high-yielding palms of these clusters and eight low-yielding palms of these clusters also indicated that palms having the GG genotype (n=13) exhibit a higher O/DM in comparison to palms having the AG genotype (n=3), specifically 12.13% higher mean O/DM (p-value=2.35E-04).

The S_00007694_00038606 SNP marker is located in candidate gene region 5 within the 3′ untranslated region of the gene encoding Cinnamoyl-CoA Reductase, specifically 19 base pairs from the stop codon thereof, and has a SNP change corresponding to “A/G.” Sequence analysis indicates to miRNA binding site.

Taken together, the data for candidate gene region 5 and the S_00007694_00038606 SNP marker therein show consistency, coherency, and biological response. For example, the S_00007694_00038606 SNP marker yielded consistent results in more than one cluster of palms, namely in five clusters, albeit with p-values <0.001 for only four of the five clusters. For the S_00007694_00038606 SNP marker, the GG genotype in particular was found to have the highest mean O/DM among the genotypes in the five clusters. In addition, similar results were obtained with respect to mesocarp per fruit, shell per fruit, and kernel per fruit, indicating coherency between the data for O/DM, M/F, S/F, and K/F and consistency in more than one cluster.

### Candidate Gene Region 6

Candidate gene region 6 includes a gene encoding 1-Aminocyclopropane-1-Carboxylate Synthase 7. Specifically, candidate gene region 6 includes a predicted transcription unit, designated SDt076123, that includes 4 predicted exons, and extends from 4 kb upstream of the transcription unit to 4 kb downstream of the transcription unit. Data from “omics” platforms, domain checks, and homology analyses indicated that the transcription unit corresponds to 1-Aminocyclopropane-1-Carboxylate Synthase 7. 1-Aminocyclopropane-1-Carboxylate Synthase 7 catalyzes the rate-limiting step in biosynthesis of ethylene, which is responsible for fruit ripening, and is encoded by members of a divergent multigene family, the genes of which are differentially regulated by various environmental and developmental factors during plant growth.

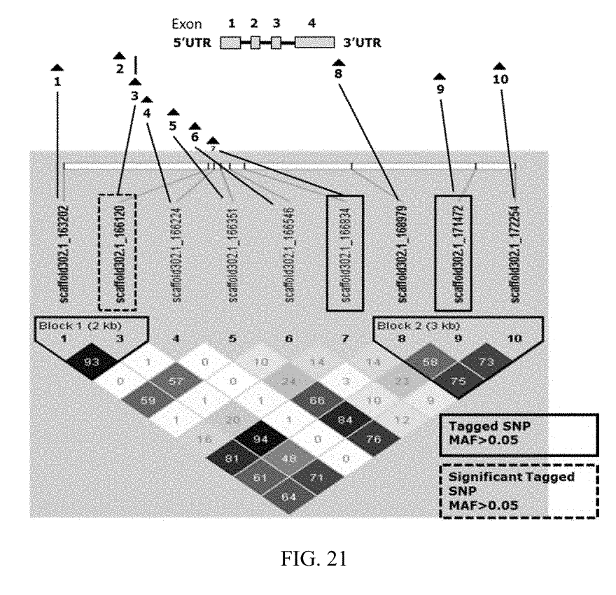

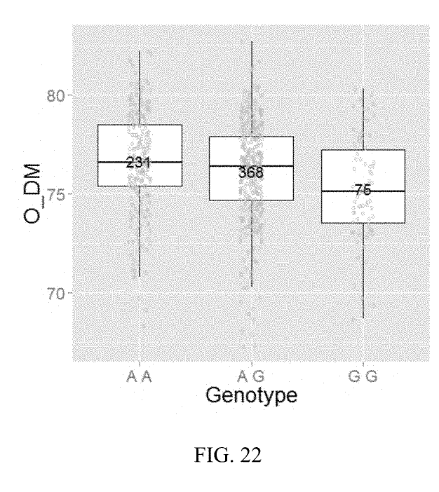

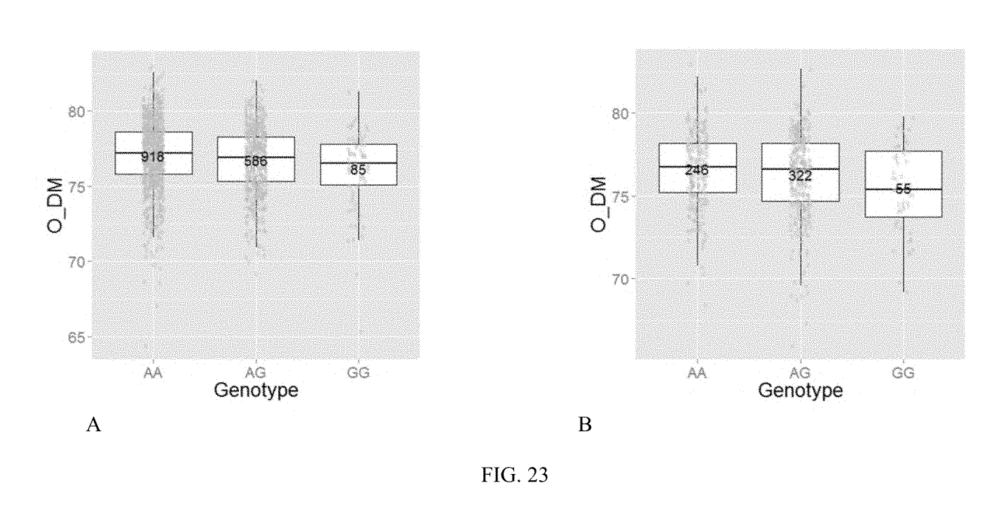

As shown in FIG. 21, genome-wide association studies revealed that candidate gene region 6 includes ten SNP markers. As shown in FIG. 22 and FIG. 23, of the ten SNP markers, one SNP marker, designated S_00000302_00166120 and having a SNP genotype major allele AG and a SNP genotype minor allele GG, is associated, after stratification and kinship correction, with the high-oil-production trait with a p-value <0.001 in the population, with respect to the Dumpy AVROS cluster. Moreover, as also shown, similar results were obtained with respect to the AVROS cluster and the Johor Labis x AVROS cluster, albeit not with a p-value <0.001.

Considering the S_00000302_00166120 SNP marker in more detail, for the Dumpy AVROS cluster, pairwise comparison of palms having the AA genotype (n=231), the AG genotype (n=368), and the GG genotype (n=75) by Student T-test indicated that palms having the AA genotype have a mean O/DM that is 0.2% higher than that of palms having the AG genotype (p-value=1.69E-02), palms having the AA genotype have a mean O/DM that is 1.5% higher than that of palms having the GG genotype (p-value=6.58E-05), and palms having the AG genotype have a mean O/DM that is 1.3% higher than that of palms having the GG genotype (p-value=5.62E-03) (FIG. 22). Similarly, for the AVROS cluster, pairwise comparison of palms having the AA genotype (n=918), the AG genotype (n=586), and the GG genotype (n=85) by Student T-test indicated that palms having the AA genotype have a mean O/DM that is 0.3% higher than that of palms having the AG genotype (p-value=6.40E-03), palms having the AA genotype have a mean O/DM that is 0.7% higher than that of palms having the GG genotype (p-value=1.07E-03), and palms having the AG genotype have a mean O/DM that is 0.4% higher than that of palms having the GG genotype (p-value=3.85E-02) (FIG. 23A). Also, for the Johor Labis x AVROS cluster, pairwise comparison of palms having the AA genotype (n=246), the AG genotype (n=322), and the GG genotype (n=55) by Student T-test indicated that palms having the AA genotype have a mean O/DM that is 0.2% higher than that of palms having the AG genotype (p-value=9.25E-02), palms having the AA genotype have a mean O/DM that is 1.3% higher than that of palms having the GG genotype (p-value=1.34E-03), and palms having the AG genotype have a mean O/DM that is 1.2% higher than that of palms having the GG genotype (p-value=2.81E-02) (FIG. 23B).

Comparative analysis of expression of the SDt076123 transcript, i.e. transcript of 1-Aminocyclopropane-1 -Carboxylate Synthase 7, mean O/DM for each of genotypes AA, AG, and GG, for eight high-yielding palms of these clusters and eight low-yielding palms of these clusters also indicated that palms having the AA genotype (n=6) exhibit a higher O/DM in comparison to palms having the AG genotype (n=6) and palms having the GG genotype (n=4), specifically 0.3% higher mean O/DM (p-value=0.58) and 2.7% higher mean O/DM (p-value=0.97), respectively.

The S_00000302_00166120 SNP marker is located in candidate gene region 6 within the 5′ untranslated region of the gene encoding 1-Aminocyclopropane-1-Carboxylate Synthase 7, specifically 728 base pairs from the ATG start codon thereof, and has a SNP change corresponding to “T>C.”

Taken together, the data for candidate gene region 6 and the S_00000302_00166120 SNP marker therein show consistency, coherency, and biological response. For example, the S_00000302_00166120 SNP marker yielded consistent results in more than one cluster of palms, namely in three clusters, albeit with p-values <0.001 for only one of the three clusters, and the AA genotype in particular was found to have the highest mean O/DM among the genotypes in all three of the clusters. Moreover, consistent results were observed regarding transcriptomics data for genotypes of the eight highest yielding palms and the eight lowest yielding palms, such that palms having the AA genotype have the highest mean O/DM.

### Candidate Gene Region 7

Candidate gene region 7 includes a gene encoding Mitochondrial Trans-2-Enoyl-CoA Reductase. Specifically, candidate gene region 7 includes a predicted transcription unit, designated SDt098109, that includes 11 predicted exons, and extends from 4 kb upstream of the transcription unit to 4 kb downstream of the transcription unit. Data from “omics” platforms, domain checks, and homology analyses indicated that the transcription unit corresponds to Mitochondrial Trans-2-Enoyl-CoA Reductase. Trans-2-Enoyl-CoA Reductase of Euglena gracilis has been reported to be useful for increasing lipid content in plants based on overexpression.

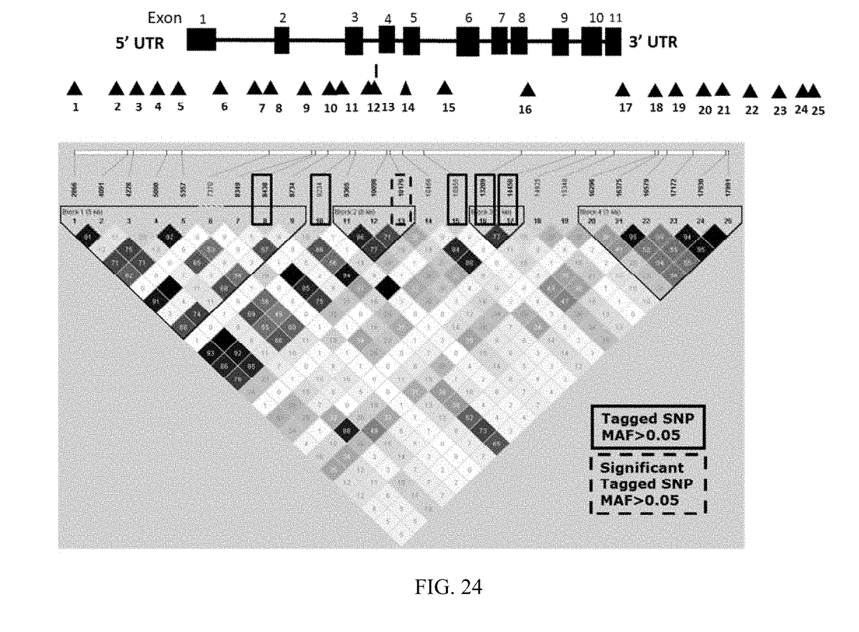

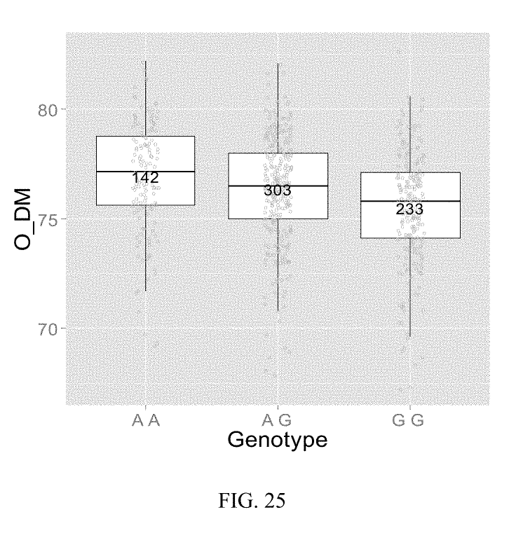

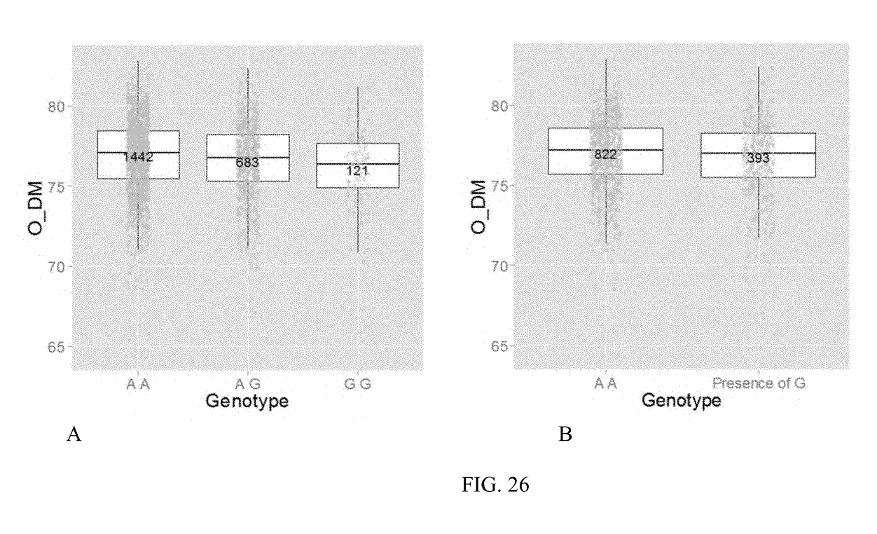

As shown in FIG. 24, genome-wide association studies revealed that candidate gene region 7 includes 25 SNP markers. As shown in FIG. 25 and FIG. 26, of the 25 SNP markers, one SNP marker, designated S_00002174_00010170 and having a SNP genotype major allele AG and a SNP genotype minor allele AA, is associated, after stratification and kinship correction, with the high-oil-production trait with a p-value <0.001 in the population, with respect to the Dumpy AVROS cluster and the UR_ALL cluster. Moreover, as also shown, similar results were obtained with respect to the UR x AVROS cluster, albeit not with a p-value <0.001.

Considering the S_00002174_00010170 SNP marker in more detail, for the Dumpy AVROS cluster, pairwise comparison of palms having the AA genotype (n=142), the AG genotype (n=303), and the GG genotype (n=233) by Student T-test indicated that palms having the AA genotype have a mean O/DM that is 0.65% higher than that of palms having the AG genotype (p-value=0.02), palms having the AA genotype have a mean O/DM that is 1.35% higher than that of palms having the AG genotype (p-value=2.98E-07), and palms having the AG genotype have a mean O/DM that is 0.7% higher than that of palms having the GG genotype (p-value=1.49E-04) (FIG. 25). Similarly, for the UR_ALL cluster, pairwise comparison of palms having the AA genotype (n=1442), the AG genotype (n=683), and the GG genotype (n=121) by Student T-test indicated that palms having the AA genotype have a mean O/DM that is 0.3% higher than that of palms having the AG genotype (p-value=2.51E-03), palms having the AA genotype have a mean O/DM that is 0.7% higher than that of palms having the GG genotype (p-value=2.14E-04), and palms having the AG genotype have a mean O/DM that is 0.4% higher than that of palms having the GG genotype (p-value=0.03) (FIG. 26A). Also, for the UR x AVROS cluster, pairwise comparison of palms having the AA genotype (n=822) and the AG genotype and the GG genotype together (n=393) by Student T-test indicated that palms having the AA genotype have a mean O/DM that is 0.2% higher than that of palms having the AG genotype and palms having the GG genotype, considered together (p-value=0.09) (FIG. 26B).

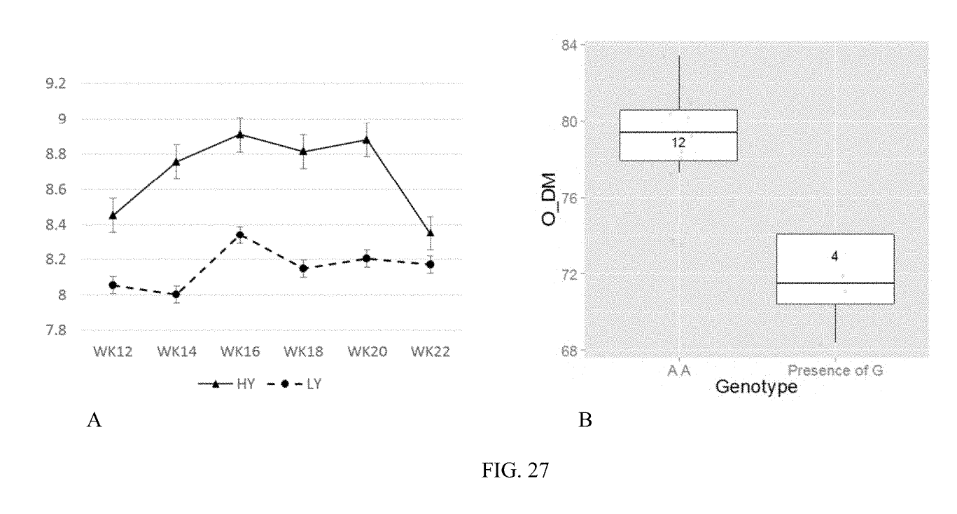

As shown in FIG. 27, comparative analysis of expression of the SDt098109 transcript, i.e. transcript of Mitochondrial Trans-2-Enoyl-CoA Reductase, and mean O/DM for each of genotypes AA and AG and GG, for eight high-yielding palms of these clusters and eight low-yielding palms of these clusters also indicated that palms having the AA genotype (n=12) exhibit a higher O/DM in comparison to palms having the AG and GG genotypes (n=4), specifically 6.6% higher mean O/DM (p-value=9.73E-04).

The S_00002174_00010170 SNP marker is located in candidate gene region 7 within an intron of the gene encoding Mitochondrial Trans-2-Enoyl-CoA Reductase, specifically 128 base pairs from exon 3 and 148 base pairs from exon 4 thereof, and has a SNP change corresponding to “G/A.”

Taken together, the data for candidate gene region 7 and the S_00002174_00010170 SNP marker therein show consistency, coherency, and biological response. For example, the S_00002174_00010170 SNP marker yielded consistent results in more than one cluster of palms, namely in three clusters, and the AA genotype in particular was found to have the highest mean O/DM among the genotypes in all three of the clusters. Moreover, consistent results were observed regarding transcriptomics data for genotypes of the eight highest yielding palms and the eight lowest yielding palms, such that palms having the AA genotype have the highest mean O/DM.

### Candidate Gene Region 8

Candidate gene region 8 includes a gene encoding Chloroplastic Biotin Carboxyl Carrier Protein of Acetyl CoA Carboxylase. Specifically, candidate gene region 8 includes a predicted transcription unit, designated SDt83215, that includes 9 predicted exons, and extends from 4 kb upstream of the transcription unit to 4 kb downstream of the transcription unit. Data from “omics” platforms, domain checks, and homology analyses indicated that the transcription unit corresponds to Chloroplastic Biotin Carboxyl Carrier Protein of Acetyl CoA Carboxylase. Chloroplastic Biotin Carboxyl Carrier Protein of Acetyl CoA Carboxylase is a component of acetyl-CoA carboxylase and plays a role in carboxylation thereby.

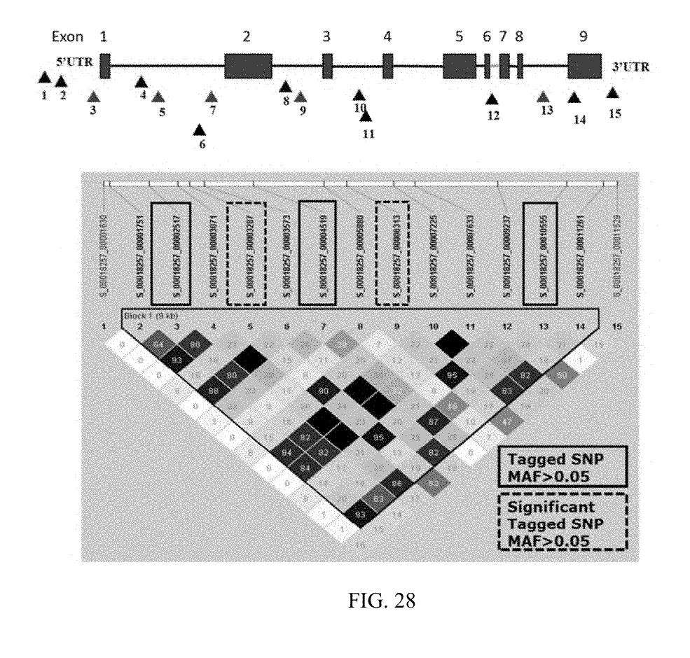

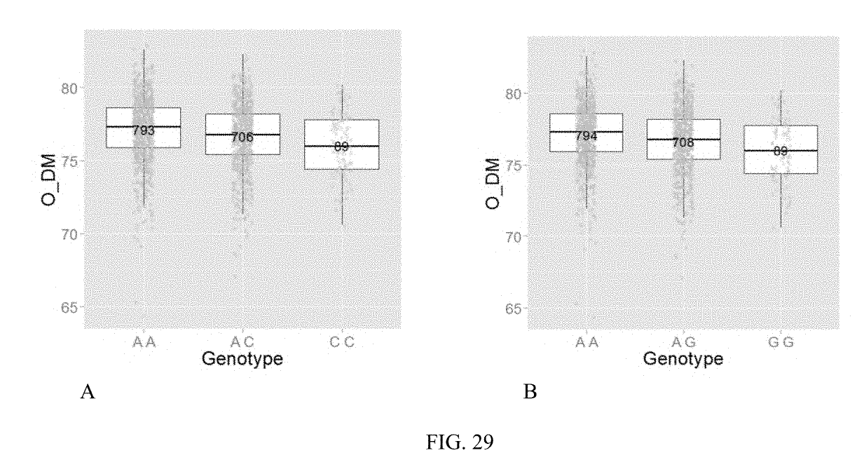

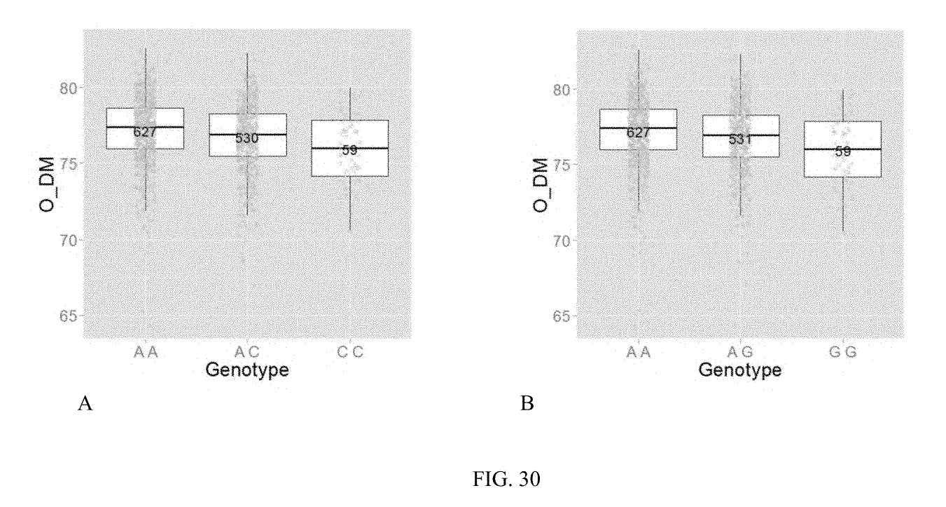

As shown in FIG. 28, genome-wide association studies revealed that candidate gene region 8 includes 15 SNP markers. As shown in FIG. 29 and FIG. 30, of the 15 SNP markers, two SNP markers, a first SNP marker designated S_00018257_00003287 and having a SNP genotype major allele AA and a SNP genotype minor allele CC, and a second SNP marker designated S_00018257_00006313 and having a SNP genotype major allele AA and a SNP genotype minor allele GG, are associated, after stratification and kinship correction, with the high-oil-production trait with a p-value <0.001 in the population, with respect to the AVROS cluster and the UR x AVROS cluster.

Considering the S_00018257_00003287 SNP marker in more detail, for the AVROS cluster, pairwise comparison of palms having the AA genotype (n=793), the AC genotype (n=706), and the CC genotype (n=89) by Student T-test indicated that palms having the AA genotype have a mean O/DM that is 0.5% higher than that of palms having the AC genotype (p-value=0.02), palms having the AA genotype have a mean O/DM that is 1.4% higher than that of palms having the CC genotype (p-value=3.00E-04), and palms having the AC genotype have a mean O/DM that is 0.9% higher than that of palms having the CC genotype (p-value=0.02) (FIG. 29A). Similarly, for the UR x AVROS cluster, pairwise comparison of palms having the AA genotype (n=627), the AC genotype (n=530), and the CC genotype (n=59) by Student T-test indicated that palms having the AA genotype have a mean O/DM that is 0.5% higher than that of palms having the AC genotype (p-value=9.47E-05), palms having the AA genotype have a mean O/DM that is 1.3% higher than that of palms having the CC genotype (p-value=3.37E-05), and palms having the AC genotype have a mean O/DM that is 0.8% higher than that of palms having the CC genotype (p-value=0.02) (FIG. 30A).

Considering the S_00018257_00006313 SNP marker in more detail, for the AVROS cluster, pairwise comparison of palms having the AA genotype (n=794), the AG genotype (n=708), and the GG genotype (n=89) by Student T-test indicated that palms having the AA genotype have a mean O/DM that is 0.5% higher than that of palms having the AG genotype (p-value=0.02), palms having the AA genotype have a mean O/DM that is 1.4% higher than that of palms having the GG genotype (p-value=3.00E-04), and palms having the AG genotype have a mean O/DM that is 0.9% higher than that of palms having the GG genotype (p-value=0.02) (FIG. 29B). Similarly, for the UR x AVROS cluster, pairwise comparison of palms having the AA genotype (n=627), the AG genotype (n=531), and the GG genotype (n=59) by Student T-test indicated that palms having the AA genotype have a mean O/DM that is 0.5% higher than that of palms having the AG genotype (p-value=8.57E-05), palms having the AA genotype have a mean O/DM that is 1.3% higher than that of palms having the GG genotype (p-value=3.22E-05), and palms having the AG genotype have a mean O/DM that is 0.8% higher than that of palms having the GG genotype (p-value=0.01) (FIG. 30B).

Similar results also were obtained with respect to oil-to-wet mesocarp and oil per palm (also termed O/P or O_P) for three other SNP markers from among the 15 SNP markers located in candidate gene region 8. Specifically, for a SNP marker designated S_00018257_00002517 and having a SNP genotype major allele AG and a SNP genotype minor allele GG, for the UR x Dumpy AVROS cluster the AA genotype yielded higher O/WM and higher O/P than the AG genotype or the GG genotype. Also, for a SNP marker designated S_00018257_00004519 and having a SNP genotype major allele AG and a SNP genotype minor allele AA, for the UR x Dumpy AVROS cluster the AA genotype yielded higher oil per hectare (also termed O/Ha) and higher O/P than the AG genotype or the GG genotype. Also, for a SNP marker designated S_00018257_000010555 and having a SNP genotype major allele AG and a SNP genotype minor allele AA, for the UR x Dumpy AVROS cluster the AA genotype yielded higher O/Ha and higher O/P than the AG genotype or the GG genotype.

The S_00018257_00003287 SNP marker is located in candidate gene region 8 within an intron of the gene encoding Chloroplastic Biotin Carboxyl Carrier Protein of Acetyl CoA Carboxylase, specifically 626 base pairs from exon 1, between exons 1 and 2, and has a SNP change corresponding to “T/G.” The S_00018257_00006313 SNP marker is located in candidate gene region 8 within an intron of the gene encoding Chloroplastic Biotin Carboxyl Carrier Protein of Acetyl CoA Carboxylase, specifically 364 base pairs from exon 3, between exons 2 and 3, and has a SNP change corresponding to “T/C.” The S_00018257_00002517 SNP marker is located in candidate gene region 8 within the promoter of the gene encoding Chloroplastic Biotin Carboxyl Carrier Protein of Acetyl CoA Carboxylase, specifically 14 base pairs from exon 1, and has a SNP change corresponding to “T/C.” The S_00018257_00004519 SNP marker is located in candidate gene region 8 within an intron of the gene encoding Chloroplastic Biotin Carboxyl Carrier Protein of Acetyl CoA Carboxylase, specifically 883 base pairs from exon 2, between exon 1 and exon 2, and has a SNP change corresponding to “G/A.” The S_00018257_000010555 SNP marker is located in candidate gene region 8 within an intron of the gene encoding Chloroplastic Biotin Carboxyl Carrier Protein of Acetyl CoA Carboxylase, specifically 609 base pairs from exon 9, between exon 8 and exon 9, and has a SNP change corresponding to “C/T.”

Taken together, the data for candidate gene region 8 and the S_00018257_00003287 SNP marker and the S_00018257_00006313 SNP marker therein show consistency, coherency, and biological response. For example, the S_00018257_00003287 SNP marker and the S_00018257_00006313 SNP marker yielded consistent results in more than one cluster of palms, namely in two clusters. For both the S_00018257_00003287 SNP marker and the S_00018257_00006313 SNP marker, the AA genotype in particular was found to have the highest mean O/DM among the genotypes in the two clusters. Moreover, similar results were obtained with respect to oil-to-wet mesocarp and oil per palm for the S_00018257_00002517 SNP marker, the S_00018257_00004519 SNP marker, and the S_00018257_000010555 SNP marker, indicating coherency between the data for O/DM, O/WM, and O/P.

### INDUSTRIAL APPLICABILITY

The methods disclosed herein are useful for predicting oil yield of a test oil palm plant, and thus for improving commercial production of palm oil.

1. A method for predicting palm oil yield of a test oil palm plant, the method comprising the steps of:
(i) determining, from a sample of a test oil palm plant of a population of oil palm plants, at least a first single nucleotide polymorphism (SNP) genotype of the test oil palm plant, the first SNP genotype corresponding to a first SNP marker, the first SNP marker (a) being located in a first candidate gene region for a high-oil-production trait and (b) being associated, after stratification and kinship correction, with the high-oil-production trait with a p-value <0.001 in the population or having a linkage disequilibrium r² value of at least 0.2 with respect to a first other SNP marker that is linked thereto and associated, after stratification and kinship correction, with the high-oil-production trait with a p-value <0.001 in the population;
(ii) comparing the first SNP genotype of the test oil palm plant to a corresponding first reference SNP genotype indicative of the high-oil-production trait in the same genetic background as the population; and
(iii) predicting palm oil yield of the test oil palm plant based on the extent to which the first SNP genotype of the test oil palm plant matches the corresponding first reference SNP genotype,
wherein the first candidate gene region is a region of the oil palm genome corresponding to:
(1) candidate gene region 1, comprising a gene encoding CBL-Interacting Protein Kinase 32 and extending from 4 kb upstream to 4 kb downstream of the gene encoding CBL-Interacting Protein Kinase 32;
(2) candidate gene region 2, comprising a gene encoding Shaggy-Related Protein Kinase and extending from 4 kb upstream to 4 kb downstream of the gene encoding Shaggy-Related Protein Kinase;
(3) candidate gene region 3, comprising a gene encoding Probable Receptor-Like Protein Kinase and extending from 4 kb upstream to 4 kb downstream of the gene encoding Probable Receptor-Like Protein Kinase;
(4) candidate gene region 4, comprising a gene encoding Tau Class Glutathione S-Transferase and extending from 4 kb upstream to 4 kb downstream of the gene encoding Tau Class Glutathione S-Transferase; or
(5) candidate gene region 5, comprising a gene encoding Cinnamoyl-CoA Reductase and extending from 4 kb upstream to 4 kb downstream of the gene encoding Cinnamoyl-CoA Reductase.

2. The method of claim 1, wherein the high-oil-production trait comprises increased oil-to-dry mesocarp.

3. The method of claim 1 or 2, wherein the population of oil palm plants comprises a Ulu Remis dura x AVROS pisifera population, a Johor Labis dura x AVROS pisifera population, a Ulu Remis dura x Dumpy AVROS pisifera population, a Dumpy AVROS population, or a combination thereof.

4. The method of claim 1, 2, or 3, wherein:
the first candidate gene region is candidate gene region 1; and
the population of oil palm plants comprises a Ulu Remis dura x AVROS pisifera population, a Johor Labis dura x AVROS pisifera population, a Ulu Remis dura x Dumpy AVROS pisifera population, or a combination thereof.

5. The method of claim 1, 2, or 3, wherein:
the first candidate gene region is candidate gene region 2; and
the population of oil palm plants comprises a Ulu Remis dura x AVROS pisifera population, a Ulu Remis dura x Dumpy AVROS pisifera population, or a combination thereof.

6. The method of claim 1, 2, or 3, wherein:
the first candidate gene region is candidate gene region 3; and
the population of oil palm plants comprises a Dumpy AVROS population.

7. The method of claim 1, 2, or 3, wherein:
the first candidate gene region is candidate gene region 4; and
the population of oil palm plants comprises a Ulu Remis dura x AVROS pisifera population, a Johor Labis dura x AVROS pisifera population, or a combination thereof.

8. The method of claim 1, 2, or 3, wherein:
the first candidate gene region is candidate gene region 5; and
the population of oil palm plants comprises a Ulu Remis dura x AVROS pisifera population.

9. The method of any one of claims 1-8, wherein the test oil palm plant is a tenera candidate agricultural production plant.

10. The method of claim 1 or 2, wherein the population of oil palm plants comprises a Ulu Remis dura x Ulu Remis dura population, a Ulu Remis dura x Johor Labis dura population, a Johor Labis dura x Johor Labis dura population, an AVROS pisifera x AVROS tenera population, an AVROS tenera x AVROS tenera population, a Dumpy AVROS pisifera x Dumpy AVROS tenera population, a Dumpy AVROS tenera x Dumpy AVROS tenera population, or a combination thereof.

11. The method of claim 1, 2, or 10, wherein the test oil palm plant is a plant for mother palm selection and propagation, a plant for introgressed mother palm selection and propagation, or a plant for pollen donor selection and propagation.

12. The method of any one of claims 1-11, wherein the test oil palm plant is a seed, a seedling, a nursery phase plant, an immature phase plant, a cell culture plant, a zygotic embryo culture plant, or a somatic tissue culture plant.

13. The method of any one of claims 1-11, wherein the test oil palm plant is a production phase plant, a mature palm, a mature mother palm, or a mature pollen donor.

14. The method of any one of claims 1-13, wherein:
step (i) further comprises determining, from the sample of the test oil palm plant, at least a second SNP genotype of the test oil palm plant, the second SNP genotype corresponding to a second SNP marker, the second SNP marker (a) being located in a second candidate gene region for a high-oil-production trait and (b) being associated, after stratification and kinship correction, with the high-oil-production trait with a p-value <0.001 in the population or having a linkage disequilibrium r² value of at least 0.2 with respect to a second other SNP marker that is linked thereto and associated, after stratification and kinship correction, with the high-oil-production trait with a p-value <0.001 in the population; and
step (ii) further comprises comparing the second SNP genotype of the test oil palm plant to a corresponding second reference SNP genotype indicative of the high-oil-production trait in the same genetic background as the population,
wherein the second candidate gene region corresponds to one of candidate gene regions 1 to 5, with the proviso that the first candidate gene region and the second candidate gene region correspond to different candidate gene regions.

15. The method of claim 14, wherein step (iii) further comprises predicting palm oil yield of the test oil palm plant based on the extent to which the second SNP genotype of the test oil palm plant matches the corresponding second reference SNP genotype.

16. The method of claim 14 or 15, wherein:
step (i) further comprises determining, from the sample of the test oil palm plant, at least a third SNP genotype to a fifth SNP genotype of the test oil palm plant, the third SNP genotype to the fifth SNP genotype corresponding to a third SNP marker to a fifth SNP marker, respectively, the third SNP marker to the fifth SNP marker (a) being located in a third candidate gene region to a fifth candidate gene region, respectively, for the high-oil-production trait and (b) being associated, after stratification and kinship correction, with the high-oil-production trait with a p-value <0.001 in the population or having a linkage disequilibrium r² value of at least 0.2 with respect to a third other SNP marker to a fifth other SNP marker, respectively, that are linked thereto and associated, after stratification and kinship correction, with the high-oil-production trait with a p-value <0.001 in the population; and
step (ii) further comprises comparing the third SNP genotype to the fifth SNP genotype of the test oil palm plant to a corresponding third reference SNP genotype to a corresponding fifth reference SNP genotype, respectively, indicative of the high-oil-production trait in the same genetic background as the population,
wherein the third candidate gene region to the fifth candidate gene region each correspond to one of candidate gene regions 1 to 5, with the proviso that the first candidate gene region to the fifth candidate gene region each correspond to different candidate gene regions.

17. The method of claim 16, wherein step (iii) further comprises predicting palm oil yield of the test oil palm plant based on the extent to which the third SNP genotype to the fifth SNP genotype of the test oil palm plant match the corresponding third reference SNP genotype to the corresponding fifth reference SNP genotype, respectively.

18. A method of selecting a high-palm-oil-yielding oil palm plant for agricultural production of palm oil, the method comprising the steps of:
(a) predicting palm oil yield of a test oil palm plant according the method of any one of claims 1-17; and
(b) field planting the test oil palm plant for agricultural production of palm oil if the palm oil yield of the test oil palm plant is predicted to be higher than average for the population based on step (a).

19. A method of selecting a high-palm-oil-yielding oil palm plant for cultivation in cell culture, the method comprising the steps of:
(a) predicting palm oil yield of a test oil palm plant according the method of any one of claims 1-17; and
(b) subjecting at least one cell of the test oil palm plant to cultivation in cell culture if the palm oil yield of the test oil palm plant is predicted to be higher than average for the population based on step (a).

20. A method of selecting a parental oil palm plant for use in breeding to obtain agricultural production plants or improved parental oil palm plants, the method comprising the steps of:
(a) predicting palm oil yield of a test oil palm plant according the method of any one of claims 1-17; and
(b) selecting the test oil palm plant for use in breeding if the palm oil yield of tenera progeny of the test oil palm plant is predicted to be higher than average for the population based on step (a).

21. A method for predicting palm oil yield of a test oil palm plant, the method comprising the steps of:
(i) determining, from a sample of a test oil palm plant of a population of oil palm plants, at least a first single nucleotide polymorphism (SNP) genotype of the test oil palm plant, the first SNP genotype corresponding to a first SNP marker, the first SNP marker (a) being located in a first candidate gene region for a high-oil-production trait and (b) being associated, after stratification and kinship correction, with the high-oil-production trait with a p-value <0.001 in the population or having a linkage disequilibrium r² value of at least 0.2 with respect to a first other SNP marker that is linked thereto and associated, after stratification and kinship correction, with the high-oil-production trait with a p-value <0.001 in the population;
(ii) comparing the first SNP genotype of the test oil palm plant to a corresponding first reference SNP genotype indicative of the high-oil-production trait in the same genetic background as the population; and
(iii) predicting palm oil yield of the test oil palm plant based on the extent to which the first SNP genotype of the test oil palm plant matches the corresponding first reference SNP genotype,
wherein the first candidate gene region is a region of the oil palm genome corresponding to:
(1) candidate gene region 6, comprising a gene encoding 1-Aminocyclopropane-1-Carboxylate Synthase 7 and extending from 4 kb upstream to 4 kb downstream of the gene encoding 1-Aminocyclopropane-1-Carboxylate Synthase 7;
(2) candidate gene region 7, comprising a gene encoding Mitochondrial Trans-2-Enoyl-CoA Reductase and extending from 4 kb upstream to 4 kb downstream of the gene encoding Mitochondrial Trans-2-Enoyl-CoA Reductase; or
(3) candidate gene region 8, comprising a gene encoding Chloroplastic Biotin Carboxyl Carrier Protein of Acetyl CoA Carboxylase and extending from 4 kb upstream to 4 kb downstream of the gene encoding Chloroplastic Biotin Carboxyl Carrier Protein of Acetyl CoA Carboxylase.

22. The method of claim 21, wherein the high-oil-production trait comprises increased oil-to-dry mesocarp.

23. The method of claim 21 or 22, wherein the population of oil palm plants comprises a Ulu Remis dura x AVROS pisifera population, a Johor Labis dura x AVROS pisifera population, a Ulu Remis dura x Dumpy AVROS pisifera population, a Dumpy AVROS population, or a combination thereof.

24. The method of claim 21, 22, or 23, wherein:
the first candidate gene region is candidate gene region 6; and
the population of oil palm plants comprises a Dumpy AVROS population.

25. The method of claim 21, 22, or 23, wherein:
the first candidate gene region is candidate gene region 7; and
the population of oil palm plants comprises a Dumpy AVROS population.

26. The method of claim 21, 22, or 23, wherein:
the first candidate gene region is candidate gene region 8; and
the population of oil palm plants comprises a Ulu Remis dura x AVROS pisifera population, a Johor Labis dura x AVROS pisifera population, or a combination thereof.

27. The method of any one of claims 21-26, wherein the test oil palm plant is a tenera candidate agricultural production plant.

28. The method of claim 21 or 22, wherein the population of oil palm plants comprises a Ulu Remis dura x Ulu Remis dura population, a Ulu Remis dura x Johor Labis dura population, a Johor Labis dura x Johor Labis dura population, an AVROS pisifera x AVROS tenera population, an AVROS tenera x AVROS tenera population, a Dumpy AVROS pisifera x Dumpy AVROS tenera population, a Dumpy AVROS tenera x Dumpy AVROS tenera population, or a combination thereof.

29. The method of claim 21, 22, or 28, wherein the test oil palm plant is a plant for mother palm selection and propagation, a plant for introgressed mother palm selection and propagation, or a plant for pollen donor selection and propagation.

30. The method of any one of claims 21-29, wherein the test oil palm plant is a seed, a seedling, a nursery phase plant, an immature phase plant, a cell culture plant, a zygotic embryo culture plant, or a somatic tissue culture plant.

31. The method of any one of claims 21-30, wherein the test oil palm plant is a production phase plant, a mature palm, a mature mother palm, or a mature pollen donor.

32. The method of any one of claims 21-30, wherein:
step (i) further comprises determining, from the sample of the test oil palm plant, at least a second SNP genotype of the test oil palm plant, the second SNP genotype corresponding to a second SNP marker, the second SNP marker (a) being located in a second candidate gene region for a high-oil-production trait and (b) being associated, after stratification and kinship correction, with the high-oil-production trait with a p-value <0.001 in the population or having a linkage disequilibrium r² value of at least 0.2 with respect to a second other SNP marker that is linked thereto and associated, after stratification and kinship correction, with the high-oil-production trait with a p-value <0.001 in the population; and
step (ii) further comprises comparing the second SNP genotype of the test oil palm plant to a corresponding second reference SNP genotype indicative of the high-oil-production trait in the same genetic background as the population,
wherein the second candidate gene region corresponds to one of candidate gene regions 6 to 8, with the proviso that the first candidate gene region and the second candidate gene region correspond to different candidate gene regions.

33. The method of claim 32, wherein step (iii) further comprises predicting palm oil yield of the test oil palm plant based on the extent to which the second SNP genotype of the test oil palm plant matches the corresponding second reference SNP genotype.

34. The method of claim 32 or 33, wherein:
step (i) further comprises determining, from the sample of the test oil palm plant, at least a third SNP genotype of the test oil palm plant, the third SNP genotype corresponding to a third SNP marker, the third SNP marker (a) being located in a third candidate gene region for a high-oil-production trait and (b) being associated, after stratification and kinship correction, with the high-oil-production trait with a p-value <0.001 in the population or having a linkage disequilibrium r² value of at least 0.2 with respect to a third other SNP marker that is linked thereto and associated, after stratification and kinship correction, with the high-oil-production trait with a p-value <0.001 in the population; and
step (ii) further comprises comparing the third SNP genotype of the test oil palm plant to a corresponding third reference SNP genotype indicative of the high-oil-production trait in the same genetic background as the population,
wherein the third candidate gene region corresponds to one of candidate gene regions 6 to 8, with the proviso that the first candidate gene region to the third candidate gene region each correspond to different candidate gene regions.

35. The method of claim 34, wherein step (iii) further comprises predicting palm oil yield of the test oil palm plant based on the extent to which the third SNP genotype of the test oil palm plant matches the corresponding third reference SNP genotype.

36. A method of selecting a high-palm-oil-yielding oil palm plant for agricultural production of palm oil, the method comprising the steps of:
(a) predicting palm oil yield of a test oil palm plant according the method of any one of claims 21-35; and
(b) field planting the test oil palm plant for agricultural production of palm oil if the palm oil yield of the test oil palm plant is predicted to be higher than average for the population based on step (a).

37. A method of selecting a high-palm-oil-yielding oil palm plant for cultivation in cell culture, the method comprising the steps of:
(a) predicting palm oil yield of a test oil palm plant according the method of any one of claims 21-35; and
(b) subjecting at least one cell of the test oil palm plant to cultivation in cell culture if the palm oil yield of the test oil palm plant is predicted to be higher than average for the population based on step (a).

38. A method of selecting a parental oil palm plant for use in breeding to obtain agricultural production plants or improved parental oil palm plants, the method comprising the steps of:
(a) predicting palm oil yield of a test oil palm plant according the method of any one of claims 21-35; and
(b) selecting the test oil palm plant for use in breeding if the palm oil yield of tenera progeny of the test oil palm plant is predicted to be higher than average for the population based on step (a).
